(19) United States
(12) Patent Application Publication
(10) Pub. No.: US 2004/0235834 A1
(43) Pub. Date: Nov. 25, 2004

(54) Thiazoles useful as inhibitors of protein kinases

(76) Inventors: Luc J. Farmer, Foxboro, MA (US); Edmund Martin Harrington, Plymouth, MA (US); Francesco G. Salituro, Marlboro, MA (US); Jian Wang, Newton, MA (US)

Correspondence Address: VERTEX PHARMACEUTICALS INC., 130 WAVERLY STREET, CAMBRIDGE, MA, 02139-4242, US

(21) Appl. No.: 10/809,944

(22) Filed: Mar. 25, 2004

Related U.S. Application Data
(60) Provisional application No. 60/457,218, filed on Mar. 25, 2003.

(51) Int. Cl. A61K 31/541; A61K 31/5377; A61K 31/506; C07D 417/14

(52) U.S. Cl. 514/227.5; 514/235.5; 514/275; 544/60; 544/124; 544/331

(57) ABSTRACT

The present invention relates to compounds useful of inhibitors of protein kinases. The invention also provides pharmaceutically acceptable compositions comprising said compounds and methods of using the compositions in the treatment of various disease, conditions, or disorders.

### CROSS-REFERENCE TO RELATED APPLICATIONS

The present application claims priority under 35 U.S.C. §119 to U.S. Provisional Application No. 60/457,218, filed Mar. 25, 2003, entitled “Thiazoles Useful as Inhibitors of Protein Kinases”, the entire contents of which is hereby incorporated by reference.

### TECHNICAL FIELD OF THE INVENTION

The present invention relates to compounds useful as inhibitors of protein kinases. The invention also provides pharmaceutically acceptable compositions comprising the compounds of the invention and methods of using the compositions in the treatment of various disorders.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. (See, Hardie, G. and Hanks, S. The Protein Kinase Facts Book, I and II, Academic Press, San Diego, Calif.: 1995). Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.). Sequence motifs have been identified that generally correspond to each of these kinase families (See, for example, Hanks, S. K., Hunter, T., FASEB J. 1995, 9, 576-596; Knighton et al., Science 1991, 253, 407-414; Hiles et al., Cell 1992, 70, 419-429; Kunz et al., Cell 1993, 73, 585-596; Garcia-Bustos et al., EMBO J. 1994, 13, 2352-2361).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there has been a substantial effort in medicinal chemistry to find protein kinase inhibitors that are effective as therapeutic agents.

Syk is a tyrosine kinase that plays a critical role in FcFRI mediated mast cell degranulation and eosinophil activation. Accordingly, Syk kinase is implicated in various allergic disorders, in particular asthma. It has been shown that Syk binds to the phosphorylated gamma chain of the FceRI receptor via N-terminal SH2 domains and is essential for downstream signaling [Taylor et al., Mol. Cell. Biol. 1995, 15, 4149].

Inhibition of eosinophil apoptosis has been proposed as a key mechanism for the development of blood and tissue eosinophilia in asthma. IL-5 and GM-CSF are upregulated in asthma and are proposed to cause blood and tissue eosinophilia by inhibition of eosinophil apoptosis. Inhibition of eosinophil apoptosis has been proposed as a key mechanism for the development of blood and tissue eosinophilia in asthma. It has been reported that Syk kinase is required for the prevention of eosinophil apoptosis by cytokines (using antisense)[Yousefi et al., J. Exp. Med. 1996,183, 1407].

The role of Syk in FcyR dependent and independent response in bone marrow derived macrophages has been determined by using irradiated mouse chimeras reconstituted with fetal liver cells from Syk −/− embryos. Syk deficient macrophages were defective in phagocytosis induced by FcyR but showed normal phagocytosis in response to complement [Kiefer et al., Mol. Cell. Biol. 1998, 18, 4209]. It has also been reported that aerosolized Syk antisense suppresses Syk expression and mediator release from macrophages [Stenton et al., J. Immunology 2000, 164, 3790].

ZAP-70 is essential for T-cell receptor signalling. Expression of this tyrosine kinase is restricted to T-cells and natural killer cells. The importance of ZAP-70 in T-cell function has been demonstrated in human patients, human T-cell lines and mice. Human patients suffering from a rare form of severe combined deficiency syndrome (SCID) possess homozygous mutations in ZAP-70 (reviewed in Elder J. of Pedriatric Hematology/Oncology 1997, 19(6), 546-550). These patients have profound immunodeficiency, lack CD8+ T-cells and have CD4+ T-cells that are unresponsive to T-cell receptor (TCR)-mediated stimulation. Following TCR activation these CD4+ cells show severe defects in Ca2+ mobilization, tyrosine phosphorylation of down-stream substrates, proliferation and IL-2 production 70 (reviewed in Elder Pedriatric Research 39, 743-748). Human Jurkat cells lacking ZAP-70 also provide important insights into the critical role of ZAP-70 in T-cell receptor signalling. A Jurkat clone (p116) with no detectable ZAP-70 protein was shown to have defects in T-cell receptor signalling which could be corrected by re-introduction of wild type ZAP-70 (Williams et al., Molecular and Cellular Biology 1998, 18 (3), 1388-1399). Studies of mice lacking ZAP-70 also demonstrate a requirement of ZAP-70 in T-cell receptor signalling. ZAP-70-deficient mice have profound defects in T-cell development and T-cell receptor signalling in thymocytes is impaired (Negishi et al., Nature 1995 376, 435-438).

The importance of the kinase domain in ZAP-70 function is demonstrated by studies of human patients and mice expressing identical mutations in the DLAARN motif within the kinase domain of ZAP-70. Inactivation of kinase activity by this mutation results in defective T-cell receptor signalling (Elder et al., J. Immunology 2001, 656-661). Catalytically inactive ZAP-70 (Lys369Arg) was also defective in restoring T-cell receptor signalling in a ZAP-70 deficient Jurkat cell clone (pll6) (Williams et al., Molecular and Cellular Biology 1998, 18 (3), 1388-1399).

Accordingly, there is a great need to develop compounds useful as inhibitors of protein kinases. In particular, it would be desirable to develop compounds that are useful as inhibitors of SYK or ZAP-70, particularly given the inadequate treatments currently available for the majority of the disorders implicated in their activation.

### SUMMARY OF THE INVENTION

It has now been found that compounds of this invention, and pharmaceutically acceptable compositions thereof, are effective as inhibitors of protein kinases. In certain embodiments, these compounds are effective as inhibitors of SYK or ZAP-70 protein kinases. These compounds have the general formula I:

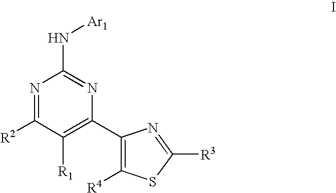

or a pharmaceutically acceptable salt thereof, wherein R¹, R², R³, R⁴, and Ar¹ are as defined below.

These compounds and pharmaceutically acceptable compositions thereof are useful for treating or preventing a variety of diseases, disorders or conditions, including, but not limited to, immunodeficiency disorders, inflammatory diseases, allergic diseases, autoimmune diseases, proliferative disorders, immunologically-mediated diseases, or respiratory disorders, to name a few. The compounds provided by this invention are also useful for the study of kinases in biological and pathological phenomena; the study of intracellular signal transduction pathways mediated by such kinases; and the comparative evaluation of new kinase inhibitors.

### DETAILED DESCRIPTION OF THE INVENTION

I. General Description of Compounds of the Invention:

The present invention relates to a compound of formula I:

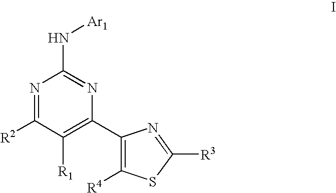

or a pharmaceutically acceptable salt thereof, wherein:

R¹ and R² are each independently R, halogen, CN, NO₂, or TR, or R¹ and R² taken together form an optionally substituted saturated, partially unsaturated, or fully unsaturated 5- or 6-membered ring having 0-3 heteroatoms independently selected from N, O, or S;

T is an optionally substituted C₁-C₄ alkylidene chain wherein up to two methylene units of T are optionally and independently replaced by O, N(R), C(O), S, SO, or SO₂;

Ar¹ is an optionally substituted ring selected from: an aryl group selected from a 5-6 membered monocyclic or an 8-10 membered bicyclic ring having 0-5 heteroatoms independently selected from nitrogen, oxygen, or sulfur; a 3-8-membered saturated or partially unsaturated monocyclic ring having 0-3 heteroatoms independently selected from nitrogen, oxygen, or sulfur; or an 8-10-membered saturated or partially unsaturated bicyclic ring system having 0-5 heteroatoms independently selected from nitrogen, oxygen, or sulfur; wherein Ar¹ is optionally substituted at one or more carbon atoms with 0-5 occurrences of —Q—R⁵, and at one or more substitutable nitrogen atoms with —R⁶ and each occurrence of R⁶ is independently R¹, —COR¹, —CO₂(C₁₋₆ aliphatic), —CON(R )₂, —SO₂N(R′)₂, or —SO₂R′;

R³ and R⁴ are each independently Z—R⁷;

each occurrence of Q and Z is independently a bond or an optionally substituted C₁-C₆ alkylidene chain wherein up to two non-adjacent methylene units of Q are optionally replaced by CO, CO₂, COCO, CONR, OCONR, NRNR, NRNRCO, NRCO, NRCO₂, NRCONR, SO, SO₂, NRSO₂, SO₂NR, NRSO₂NR, O, S, or NR;

each occurrence of R⁵ and R⁷ is independently R′, halogen, NO₂, CN, OR′, SR′, N(R′)₂, NR′C(O)R′, NR′C(O)N(R′)₂, NR′CO₂R′, C(O)R′, CO₂R′, OC(O)R′, C(O)N(R′)₂, OC(O)N(R′)₂, SOR′, SO₂R′, SO₂N(R′)₂, NR′SO₂R′, NR′SO₂N(R′)₂, PO(OR′)₂, C(O)C(O)R′, or C(O)CH₂C(O)R′; and

each occurrence of R is independently hydrogen or an optionally substituted C₁₋₆ aliphatic group; and each occurrence of R′ is independently hydrogen or an optionally substituted group selected from C₁₋₈ aliphatic, C₆₋₁₀ aryl, a heteroaryl ring having 5-10 ring atoms, or a heterocyclyl ring having 3-10 ring atoms, or wherein two occurrences of R taken together, R and R′ taken together, or two occurrences of R′ taken together, form an optionally substituted saturated, partially unsaturated, or fully unsaturated 3-8 membered ring having 0-3 heteroatoms independently selected from nitrogen, oxygen, or sulfur; provided that:

when R¹ and R² are both hydrogen, R³ is hydrogen, R⁴ is CN, or

when R¹ and R² are both hydrogen, R³ is NH₂, R⁴ is CN,

then Ar¹ is not phenyl or pyridyl substituted with one or two occurrences of Cl, Me, CH₂NRR′, C(O)NRR′, or SO₂NRR′, wherein R and R′ taken together form an optionally substituted saturated 6- or 7-membered ring having 1 or 2 heteroatoms independently selected from nitrogen or oxygen.

2. Compounds and Definitions:

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. In some embodiments, “cycloaliphatic” (or “carbocycle” or “cycloalkyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroaliphatic”, as used herein, means aliphatic groups wherein one or two carbon atoms are independently replaced by one or more of oxygen, sulfur, nitrogen, phosphorus, or silicon. Heteroaliphatic groups may be substituted or unsubstituted, branched or unbranched, cyclic or acyclic, and include “heterocycle”, “heterocyclyl”, “heterocycloaliphatic”, or “heterocyclic” groups.

The term “heterocycle”, “heterocyclyl”, “heterocycloaliphatic”, or “heterocyclic” as used herein means non-aromatic, monocyclic, bicyclic, or tricyclic ring systems in which one or more ring members is an independently selected heteroatom. In some embodiments, the “heterocycle”, “heterocyclyl”, “heterocycloaliphatic”, or “heterocyclic” group has three to fourteen ring members in which one or more ring members is a heteroatom independently selected from oxygen, sulfur, nitrogen, or phosphorus, and each ring in the system contains 3 to 7 ring members.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

The term “alkoxy”, or “thioalkyl”, as used herein, refers to an alkyl group, as previously defined, attached to the principal carbon chain through an oxygen (“alkoxy”) or sulfur (“thioalkyl”) atom.

The terms “haloalkyl”, “haloalkenyl” and “haloalkoxy” means alkyl, alkenyl or alkoxy, as the case may be, substituted with one or more halogen atoms. The term “halogen” means F, Cl, Br, or I.

The term “aryl” used alone or as part of a larger moiety as in “aralkyl”, “aralkoxy”, or “aryloxyalkyl”, refers to monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring”. The term “aryl” also refers to heteroaryl ring systems as defined hereinbelow.

The term “heteroaryl”, used alone or as part of a larger moiety as in “heteroaralkyl” or “heteroarylalkoxy”, refers to monocyclic, bicyclic, and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”.

An aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl or heteroaryl group are selected from halogen; —R^{o}; —OR^{o}; —SR^{o}; 1,2-methylene-dioxy; 1,2-ethylenedioxy; phenyl (Ph) optionally substituted with R^{o}; —O(Ph) optionally substituted with R^{o}; —(CH₂)₁₋₂(Ph), optionally substituted with R^{o}; —CH═CH(Ph), optionally substituted with R^{o}; —NO₂; —CN; —N(R^{o})₂; —NR^{o}C(O)R^{o}; —NR^{o}C(O)N(R^{o})₂; 'NR^{o}CO₂R^{o}; —NR^{o}NR^{o}C(O)R^{o}; —NR^{o}NR^{o}C(O)N(R^{o})₂; —NR^{o}NR^{o}CO₂R^{o}; —C(O)C(O)R^{o}; —C(O)CH₂C(O)R^{o}; —CO₂R^{o}; —C(O)R^{o}; —C(O)N(R^{o})₂; —OC(O)N(R^{o})₂; —S(O)₂R^{o}; —SO₂N(R^{o})₂; —S(O)R^{o}; —NR^{o}SO₂N(R^{o})₂; —NR^{o}SO₂R^{o}; —C(═S)N(R^{o})₂; —C(═NH)—N(R^{o})₂; or —(CH₂)₀₋₂NHC(O)R^{o} wherein each independent occurrence of R^{o} is selected from hydrogen, optionally substituted C₁₋₆ aliphatic, an unsubstituted 5-6 membered heteroaryl or heterocyclic ring, phenyl, —O(Ph), or —CH₂(Ph), or, notwithstanding the definition above, two independent occurrences of R^{o}, on the same substituent or different substituents, taken together with the atom(s) to which each R^{o} group is bound, form a 3-8-membered cycloalkyl, heterocyclyl, aryl, or heteroaryl ring having 0-3 heteroatoms independently selected from nitrogen, oxygen, or sulfur. Optional substituents on the aliphatic group of R^{o} are selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, wherein each of the foregoing C₁₋₄aliphatic groups of R^{o} is unsubstituted.

An aliphatic or heteroaliphatic group, or a non-aromatic heterocyclic ring may contain one or more substituents. Suitable substituents on the saturated carbon of an aliphatic or heteroaliphatic group, or of a non-aromatic heterocyclic ring are selected from those listed above for the unsaturated carbon of an aryl or heteroaryl group and additionally include the following: ═O, ═S, ═NNHR, ═NN(R*)₂, ═NNHC(O)R*, ═NNHCO₂(alkyl), ═NNHSO₂(alkyl), or ═NR, where each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R* is unsubstituted.

Optional substituents on the nitrogen of a non-aromatic heterocyclic ring are selected from —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, or —NR⁺SO₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —(CH₂)₁₋₂(Ph); optionally substituted —CH═CH(Ph); or an unsubstituted 5-6 membered heteroaryl or heterocyclic ring having one to four heteroatoms independently selected from oxygen, nitrogen, or sulfur, or, notwithstanding the definition above, two independent occurrences of R⁺, on the same substituent or different substituents, taken together with the atom(s) to which each R⁺ group is bound, form a 3-8-membered cycloalkyl, heterocyclyl, aryl, or heteroaryl ring having 0-3 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R⁺ is unsubstituted.

The term “alkylidene chain” refers to a straight or branched carbon chain that may be fully saturated or have one or more units of unsaturation and has two points of attachment to the rest of the molecule.

As detailed above, in some embodiments, two independent occurrences of R^{o} (or R⁺, or any other variable similarly defined herein), are taken together together with the atom(s) to which each variable is bound to form a 3-8-membered cycloalkyl, heterocyclyl, aryl, or heteroaryl ring having 0-3 heteroatoms independently selected from nitrogen, oxygen, or sulfur. Exemplary rings that are formed when two independent occurrences of R^{o} (or R⁺, or any other variable similarly defined herein) are taken together with the atom(s) to which each variable is bound include, but are not limited to the following: a) two independent occurrences of R^{o} (or R⁺, or any other variable similarly defined herein) that are bound to the same atom and are taken together with that atom to form a ring, for example, N(R^{o})₂, where both occurrences of R^{o} are taken together with the nitrogen atom to form a piperidin-1-yl, piperazin-1-yl, or morpholin-4-yl group; and b) two independent occurrences of R^{o} (or R⁺, or any other variable similarly defined herein) that are bound to different atoms and are taken together with both of those atoms to form a ring, for example where a phenyl group is substituted with two occurrences of OR^{o}

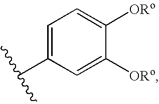

these two occurrences of R^{o} are taken together with the oxygen atoms to which they are bound to form a fused 6-membered oxygen containing ring:

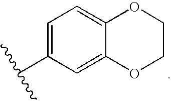

It will be appreciated that a variety of other rings can be formed when two independent occurrences of R^{o} (or R⁺, or any other variable similarly defined herein) are taken together with the atom(s) to which each variable is bound and that the examples detailed above are not intended to be limiting.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

3. Description of Exemplary Compounds:

As described generally above for compounds of general formula I, Ar¹ is an optionally substituted ring selected from: an aryl group selected from a 5-6 membered monocyclic or an 8-10 membered bicyclic ring having 0-5 heteroatoms independently selected from nitrogen, oxygen, or sulfur; a 3-8-membered saturated or partially unsaturated monocyclic ring having 0-3 heteroatoms independently selected from nitrogen, oxygen, or sulfur; or an 8-10-membered saturated or partially unsaturated bicyclic ring system having 0-5 heteroatoms independently selected from nitrogen, oxygen, or sulfur, wherein Ar¹ is optionally substituted at one or more carbon atoms with 0-5 occurrences of —Q—R⁵, and at one or more substitutable nitrogen atoms with —R⁶.

Preferred Ar¹ groups of formula I are optionally substituted rings selected from:

(a) a phenyl, indanyl, or naphthyl ring;

(b) a 5-6 membered heterocyclic ring having 1-3 heteroatoms independently selected from nitrogen, oxygen, or sulfur; or (c) a 5-6 membered monocyclic or 9-10 membered bicyclic heteroaryl ring having 1-3 heteroatoms independently selected from oxygen, nitrogen, or sulfur.

More preferred Ar¹ groups of formula I are optionally substituted rings selected from:

(a) a phenyl ring;

(b) a 5-6 membered heterocyclic ring having 1-3 heteroatoms independently selected from nitrogen, oxygen, or sulfur; or (c) a 5-6 membered monocyclic heteroaryl ring having 1-3 heteroatoms independently selected from oxygen, nitrogen, or sulfur.

In still other embodiments, Ar¹ groups of formula I are optionally substituted rings selected from any one of a-bb:

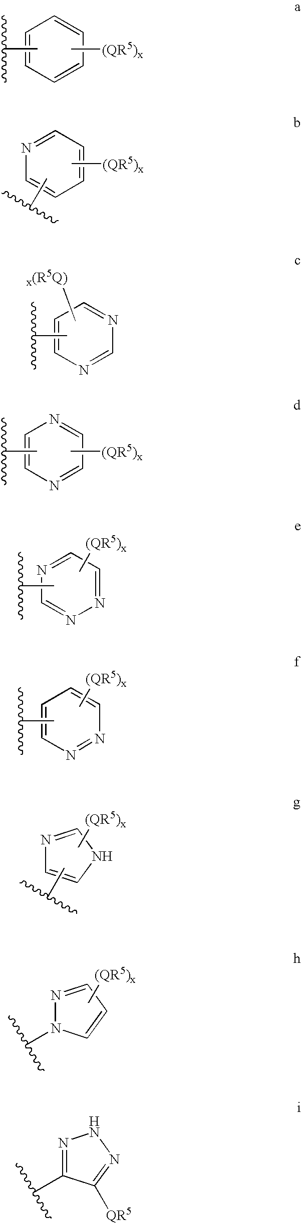

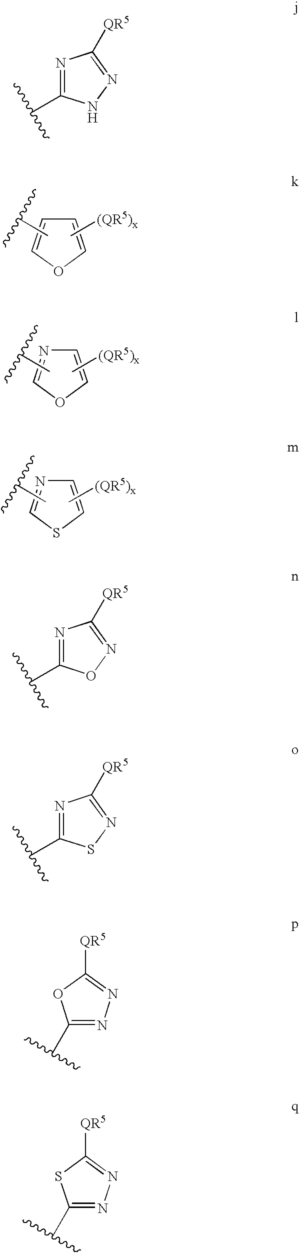

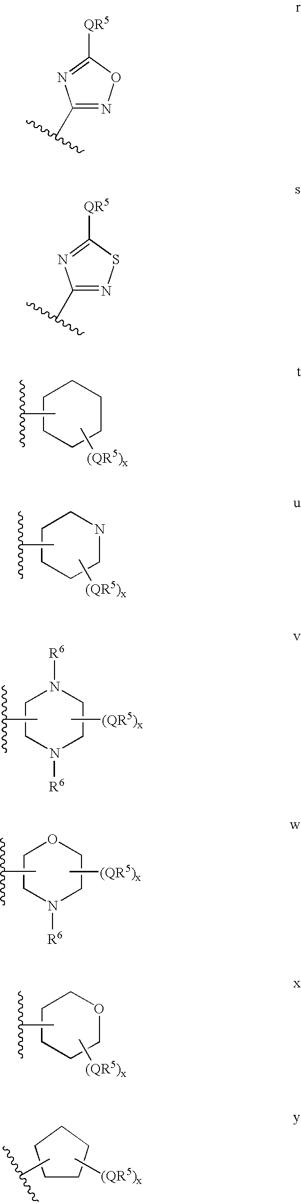

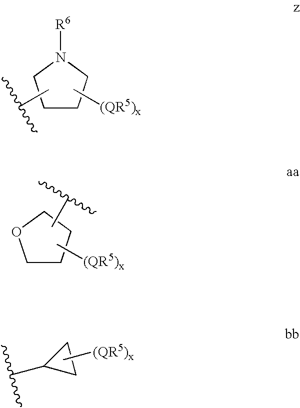

wherein x is 0-5.

Most preferred Ar¹ rings are phenyl, pyrimidinyl, or pyridyl.

As described generally above for compounds of general formula I, Ar¹ is optionally substituted with up to 5 independent occurrences of Q—R⁵, wherein each occurrence of Q is independently a bond or is an optionally substituted C₁-C₆ alkylidene chain wherein up to two non-adjacent methylene units of Q are optionally replaced by CO, CO₂, COCO, CONR, OCONR, NRNR, NRNRCO, NRCO, NRCO₂, NRCONR, SO, SO₂, NRSO₂, SO₂NR, NRSO₂NR, O, S, or NR; and each occurrence of R⁵ is independently R′, halogen, NO₂, CN, OR′, SR′, N(R′)₂, NR′C(O)R′, NR′C(O)N(R′)₂, NR′CO₂R′, C(O)R′, CO₂R′, OC(O)R′, C(O)N(R′)₂, OC(O)N(R′)₂, SOR′, SO₂R′, SO₂N(R′)₂, NR′SO₂R′, NR′SO₂N(R′)₂, PO(OR′)₂, C(O)C(O)R′, or C(O)CH₂C(O)R′. In preferred embodiments, x is 0, 1, 2, or 2. In other preferred embodiments, x is 1, 2, or 3.

In yet other preferred embodients, Q is independently a bond or is an optionally substituted C₁-C₄ alkylidene chain wherein up to two non-adjacent methylene units of Q are optionally replaced by CO, CO₂, CONR, OCONR, NRCO, NRCO₂, NRSO₂, SO₂NR, O, S, or NR; and each occurrence of R⁵ is independently selected from R′, halogen, NO₂, CN, OR′, SR′, N(R′)₂, NR′C(O)R′, NR′C(O)N(R′)₂, NR′CO₂R′, C(O)R′, CO₂R′, OC(O)R′, C(O)N(R′)₂, OC(O)N(R′)₂, SOR′, SO₂R′, SO₂N(R′)₂, NR′SO₂R′, NR′SO₂N(R′)₂, PO(OR′)₂, C(O)C(O)R′, or C(O)CH₂C(O)R′, and x is 0, 1, 2, or 3. In preferred embodiments, x is 1, 2, or 3.

Preferred Q—R⁵ substituents on Ar¹ are CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or two adjacent occurrences of Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur.

More preferred Q—R⁵ substituents on Ar¹ are fluoro, iodo, chloro, bromo, COCH₃, CO₂CH₃, C₁₋₄alkyl (for example, methyl, ethyl, propyl, cyclopropyl, n-butyl, cyclobuyl, or t-butyl), NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₋₄alkyl), CH₂NHCO(C₁₋₄alkyl), CN, CH₂CN, OH, C₁₋₄alkoxy (for example, OCH₃, OCH₂CH₃, O(CH₂)₂CH₃, or O(CH₂)₃CH₃), optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur. Other preferred Ar¹ substituents are those substituents where two adjacent occurrences of Q—R⁵, taken together with the atoms to which they are bound, and include a fused optionally substituted saturated, partially unsaturated, or fully unsaturated 5- or 6-membered ring having 0-3 heteroatoms selected from nitrogen, oxygen, or sulfur. In more preferred embodiments, these fused substituents formed by two adjacent occurrences of Q—R⁵ include an optionally substituted group selected from methylenedioxy, ethylenedioxy, propylenedioxy, thiazolyl, oxazolyl, pyrrolyl, pyrazolyl, imidazolyl, phenyl, pyridyl, pyrimidinyl, furyl, thiophene, pyran, pyrrolidinyl, piperidinyl, piperazinyl, or morpholinyl.

In yet other embodiments, Ar¹ is phenyl and is substituted with two occurrences (x =2) of Q—R⁵ and Ar¹ is:

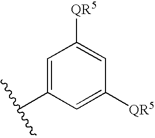

wherein each occurrence of QR⁵ is independently CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂. In certain preferred embodiments, each occurrence of QR⁵ is independently fluoro, iodo, chloro, bromo, COCH₃, CO₂CH₃, C₁₋₄alkyl (for example, methyl, ethyl, propyl, cyclopropyl, n-butyl, cyclobuyl, or t-butyl), NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₄alkyl), CH₂NHCO(C₁₋₄alkyl), CN, CH₂CN, OH, C₁₋₄alkoxy (for example, OCH₃, OCH₂CH₃, O(CH₂)₂CH₃, or O(CH₂)₃CH₃), optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur. In still other preferred embodiments, both occurrences of QR⁵ are methyl. In yet other preferred embodiments, at least one occurrence of QR⁵ is CF₃.

In yet other embodiments, Ar¹ is phenyl and is substituted with three occurrences (x=3) of Q—R⁵ and Ar¹ is:

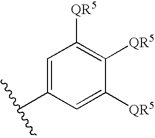

wherein each occurrence of QR⁵ is independently CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂ In certain preferred embodiments, each occurrence of QR⁵ is independently fluoro, iodo, chloro, bromo, COCH₃, CO₂CH₃, C₁₋₄alkyl (for example, methyl, ethyl, propyl, cyclopropyl, n-butyl, cyclobuyl, or t-butyl), NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₋₄alkyl), CH₂NHCO(C₁₋₄alkyl), CN, CH₂CN, OH, C₁₋₄alkoxy (for example, OCH₃, OCH₂CH₃, O(CH₂)₂CH₃, or O(CH₂)₃CH₃), optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur. In still other preferred embodiments, each occurrence of QR⁵ is independently fluoro, iodo, chloro, bromo, COCH₃, CO₂CH₃, C₁₋₄alkyl (for example, methyl, ethyl, propyl, cyclopropyl, n-butyl, cyclobuyl, or t-butyl), NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₋₄alkyl), CH₂NHCO(C₁₋₄alkyl), CN, CH₂CN, OH, optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur.

It will be appreciated that any of the Q—R⁵ substituents described above and herein are also optionally further substituted with one or more groups independently selected from R, OR, N(R)₂, SO₂R, halogen, NO₂, CN, SR, SO₂N(R)₂, CO₂R, C(O)R, or oxo. In more preferred embodiments, each of the Q—R⁵ groups described above are also optionally further substituted with one or two groups independently selected from methyl, ethyl, t-butyl, fluoro, chloro, bromo, oxo, CF₃, OMe, OEt, CN, SO₂Me, SO₂NH₂, NH₂, NHMe, N(Me)₂, SMe, SEt, OH, C(O)Me, NO₂, or CH₂OH.

As described generally above for compounds of general formula I, R¹ and R² are each independently R, halogen, CN, NO₂, or TR, or R¹ and R² taken together form an optionally substituted saturated, partially unsaturated, or fully unsaturated 5- or 6-membered ring having 0-3 heteroatoms independently selected from N, O, or S. Preferred R¹ and R² groups of formula I are hydrogen, N(R)₂, SR, OR, or TR, or R¹ and R², taken together form an optionally substituted saturated, partially unsaturated, or fully unsaturated 5-membered ring having 0-2 heteroatoms independently selected from N, O, or S. More preferred R¹ and R² groups are hydrogen, OH, CH₃, CH₂CH₃, OCH₃, CH₂OH, CH₂OCH₃, CH₂NH₂, CH₂NHCH₃, NH₂, or CH₂NH₂, or R¹ and R², taken together, form a fused optionally substituted pyrrolyl, pyrazolyl, or imidazolyl ring. Still other preferred groups include hydrogen, NH₂, or CH₂NH₂.

As described generally above for compounds of formula I, R³ and R⁴ are each independently Z—R⁷, wherein Z is an optionally substituted C₁₋₆alkylidene chain wherein up to three non-adjacent methylene units are optionally replaced by CO, CO₂, COCO, CONR, OCONR, NRNR, NRNRCO, NRCO, NRCO₂, NRCONR, SO, SO₂, NRSO₂, SO₂NR, NRSO₂NR, O, S, or NR, and each occurrence of R⁷ is independently R′, halogen, NO₂, CN, OR′, SR′, N(R′)₂, NR′C(O)R′, NR′C(O)N(R′)₂, NR′CO₂R′, C(O)R′, CO₂R′, OC(O)R′, C(O)N(R′)₂, OC(O)N(R′)₂, SOR′, SO₂R′, SO₂N(R′)₂, NR′SO₂R′, NR′SO₂N(R′)₂, PO(OR′)₂, C(O)C(O)R′, or C(O)CH₂C(O)R′.

In preferred embodiments R³ and R⁴ are each independently Z—R⁷ wherein Z is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′.

In other preferred embodiments, R³ and R⁴ are each independently hydrogen, CN, halogen, OH, SH, NH₂, CO₂H, COH, CONH₂, SO₂NH₂, NO₂, (CH₂)ₙNRR⁷, wherein R and R⁷, taken together with the nitrogen atom to which they are bound form an optionally substituted 3-8-membered saturated or partially unsaturated ring having 1-3 heteroatoms selected from nitrogen, oxygen, or sulfur.

In still other preferred embodiments, one of R³ or R⁴ is hydrogen, and the other of R³ or R⁴ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is hydrogen, (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur. In some embodiments, for compounds described directly above, R³is hydrogen. In other embodiments, for compounds described directly above, R⁴is hydrogen.

In yet other preferred embodiments, R³ or R⁴ are each independently hydrogen, (CH₂)ₙOR⁷, (CH₂)ₙNRR⁷, (CH₂)ₙCH₃, (CH₂)ₙSR⁷, (CH₂)ₙC(O)R⁷, or (CH₂)ₙC(O)R⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, wherein n is 0 or 1 and m is 0 or 1. In some preferred embodiments, R³ is hydrogen, and R⁴ is (CH₂)ₙOR⁷, (CH₂)ₙNRR⁷, (CH₂)ₙCH₃, (CH₂)ₙSR⁷, (CH₂)ₙC(O)R⁷, or (CH₂)ₙC(O)R⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1 and m is 0 or 1. In other preferred embodiments, R⁴ is hydrogen and R³ is (CH₂)ₙOR⁷, (CH₂)ₙNRR⁷, (CH₂)ₙCH₃, (CH₂)ₙSR⁷, (CH₂)ₙC(O)R⁷, or (CH₂)ₙC(O)R⁷, R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, wherein n is 0 or 1 and m is 0 or 1.

The present invention additionally provides compounds wherein at least one of R³ or R⁴ is methyl and compounds have one of formulas I-A-i or I-A-ii:

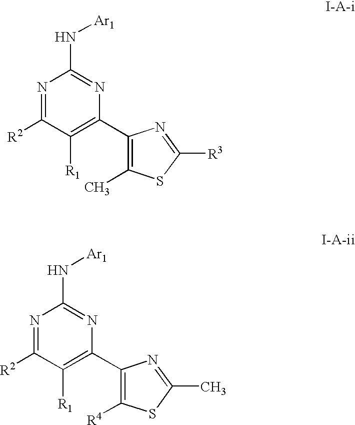

In other preferred embodiments at least one of R³ or R⁴ is (CH₂)ₙNRR⁷ and compounds have one of formulas I-B-i or I-B-ii:

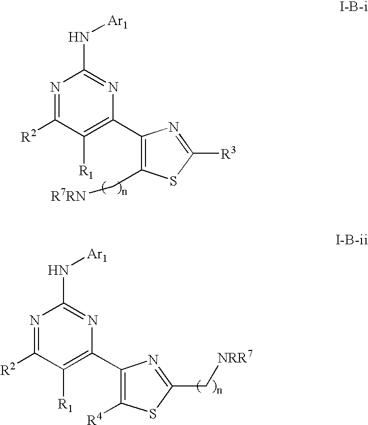

In other preferred embodiments at least one of R³ or R⁴ is (CH₂)ₙOR⁷ and compounds have one of formulas I-C-i or I-C-ii:

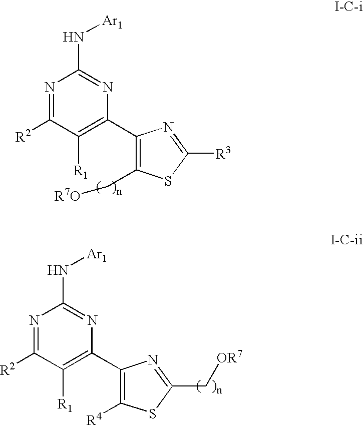

In yet other preferred embodiments both R³ and R⁴ are methyl and compounds have formula I-D-i, or R³ and R⁴ are both hydrogen and compounds have formula I-E-i:

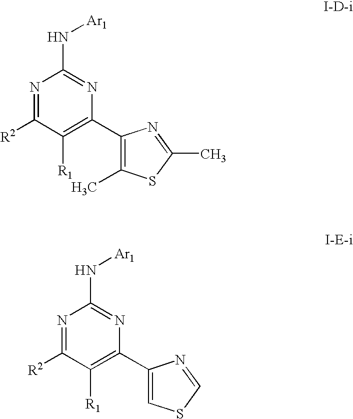

In still other preferred embodiments one of R³ or R⁴ is C(O)R⁷ and compounds have one of formulas I-F-i or I-F-ii:

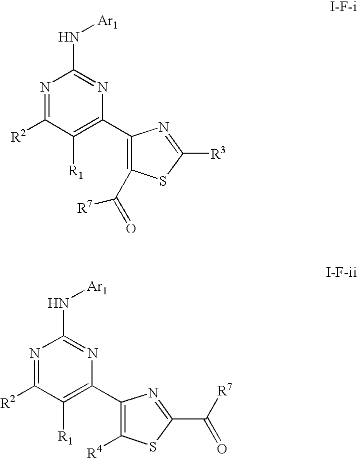

In general, for compounds of formulas I-A-i, I-A-ii, I-B-i, I-B-ii, I-C-i, I-C-ii, I-D-i, I-E-i, I-F-i, or I-F-ii, Ar¹ is an optionally substituted ring selected from: an aryl group selected from a 5-6 membered monocyclic or an 8-10 membered bicyclic ring having 0-5 heteroatoms independently selected from nitrogen, oxygen, or sulfur; a 3-8-membered saturated or partially unsaturated monocyclic ring having 0-3 heteroatoms independently selected from nitrogen, oxygen, or sulfur; or an 8-10-membered saturated or partially unsaturated bicyclic ring system having 0-5 heteroatoms independently selected from nitrogen, oxygen, or sulfur, wherein Ar¹ is optionally substituted at one or more carbon atoms with 0-5 occurrences of —Q—R⁵, and at one or more substitutable nitrogen atoms with —R⁶.

Preferred Ar¹ groups for compounds of formulas I-A-i, I-A-ii, I-B-i, I-B-ii, I-C-i, I-C-ii, I-D-i, I-E-i, I-F-i, or I-F-ii are optionally substituted rings selected from:

(a) a phenyl, indanyl, or naphthyl ring;

(b) a 5-6 membered heterocyclic ring having 1-3 heteroatoms independently selected from nitrogen, oxygen, or sulfur; or

(c) a 5-6 membered monocyclic or 9-10 membered bicyclic heteroaryl ring having 1-3 heteroatoms independently selected from oxygen, nitrogen, or sulfur.

More preferred Ar¹ groups for compounds of formulas I-A-i, I-A-ii, I-B-i, I-B-ii, I-C-i, I-C-ii, I-D-i, I-E-i, I-F-i, or I-F-ii are optionally substituted rings selected from:

(a) a phenyl ring;

(b) a 5-6 membered heterocyclic ring having 1-3 heteroatoms independently selected from nitrogen, oxygen, or sulfur; or

(c) a 5-6 membered monocyclic heteroaryl ring having 1-3 heteroatoms independently selected from oxygen, nitrogen, or sulfur.

In still other embodiments, preferred Ar¹ groups for compounds of formulas I-A-i, I-A-ii, I-B-i, I-B-ii, I-C-i, I-C-ii, I-D-i, I-E-i, I-F-i, or I-F-ii are optionally substituted rings selected from any one of a-bb:

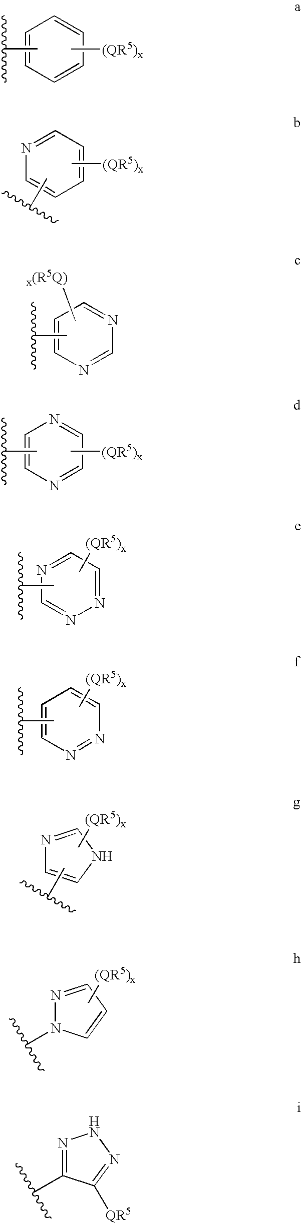

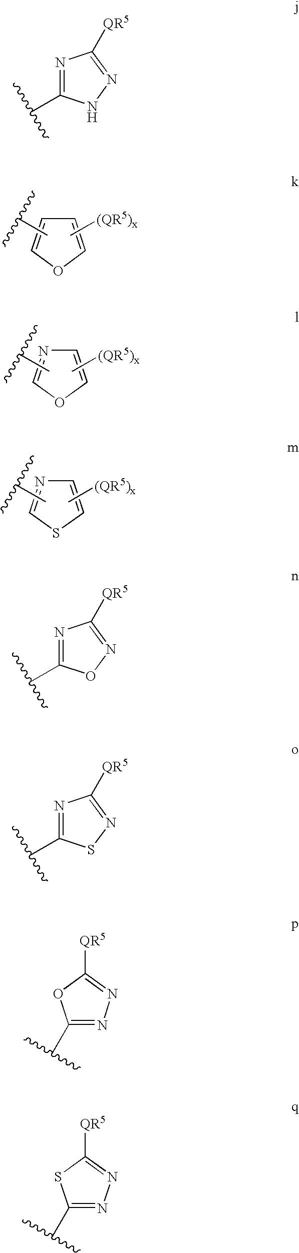

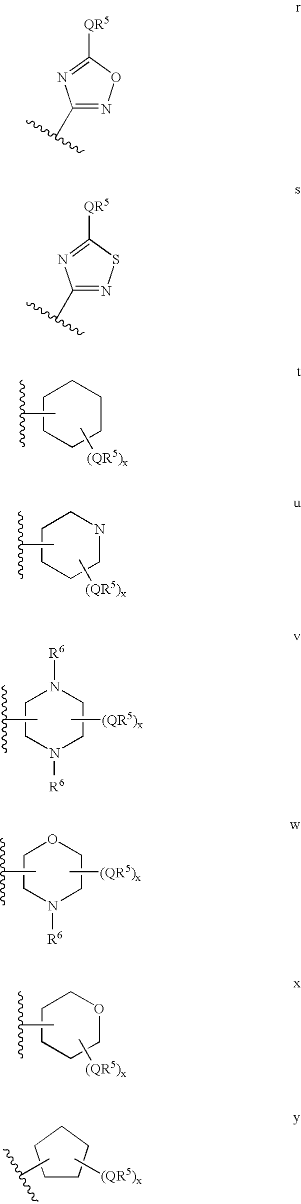

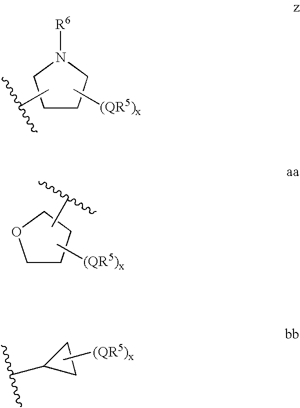

wherein Q and R⁵ are as defined generally above and in subsets herein, and x is 0-5.

Preferred Ar¹ rings for compounds of formulas I-A-i, I-A-ii, I-B-i, I-B-ii, I-C-i, I-C-ii, I-D-i, I-E-i, I-F-i, or I-F-ii are phenyl, pyrimidinyl, or pyridyl.

In preferred embodiments, for compounds of formulas I-A-i, I-A-ii, I-B-i, I-B-ii, I-C-i, I-C-ii, I-D-i, I-E-i, I-F-i, or I-F-ii, Ar¹ is optionally substituted phenyl and compounds have one of formulas I-A-i, I-A-ii, I-B-i, I-B-ii, I-C-i, I-C-ii, I-D-i, I-E-i, I-F-i, or I-F-ii:

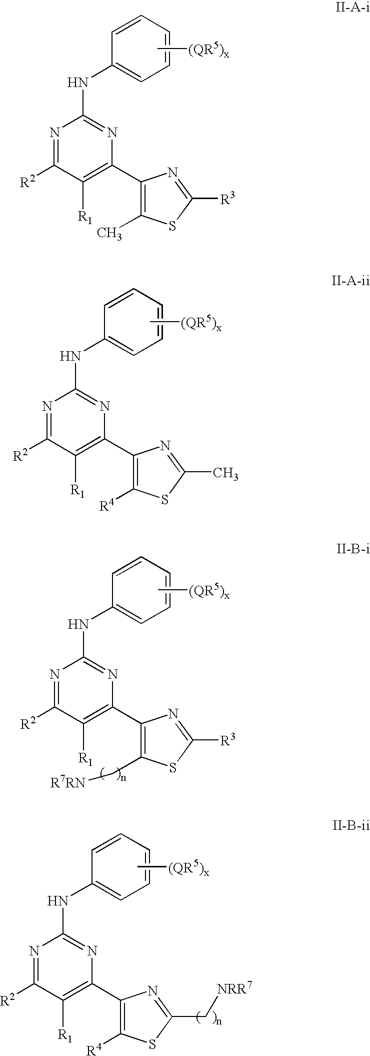

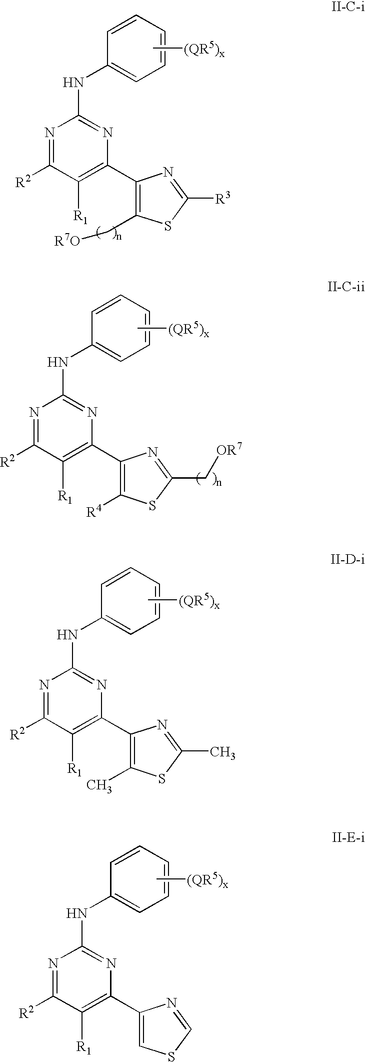

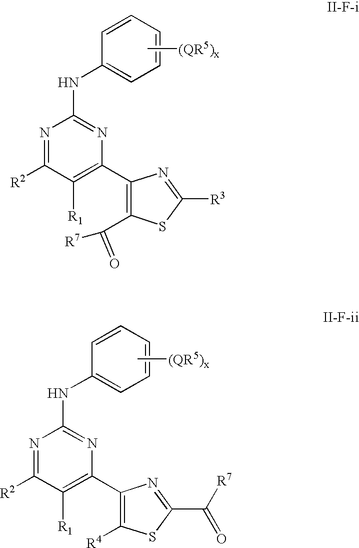

where x is 0-5.

As described generally above, Ar¹ is optionally substituted with up to 5 independent occurrences of Q—R⁵, wherein each occurrence of Q is independently a bond or is an optionally substituted C₁-C₆ alkylidene chain wherein up to two non-adjacent methylene units of Q are optionally replaced by CO, CO₂, COCO, CONR, OCONR, NRNR, NRNRCO, NRCO, NRCO₂, NRCONR, SO, SO₂, NRSO₂, SO₂NR, NRSO₂NR, O, S, or NR; and each occurrence of R⁵ is independently selected from R′, halogen, NO₂, CN, OR′, SR′, N(R′)₂, NR′C(O)R′, NR′C(O)N(R′)₂, NR′CO₂R′, C(O)R′, CO₂R′, OC(O)R′, C(O)N(R′)₂, OC(O)N(R′)₂, SOR′, SO₂R′, SO₂N(R′)₂, NR′SO₂R′, NR′SO₂N(R′)₂, PO(OR′)₂, C(O)C(O)R′, or C(O)CH₂C(O)R′. In preferred embodiments, x is 0, 1, 2, or 3. In other preferred embodiments, x is 1, 2, or 3.

In preferred embodients, for compounds of formulas I-A-i, I-A-ii, I-B-i, I-B-ii, I-C-i, I-C-ii, I-D-i, I-E-i, I-F-i, I-F-ii, II-A-i, II-A-ii, II-B-i, II-B-ii, II-C-i, II-C-ii, II-D-i, II-E-i, II-F-i, or II-F-ii, Q is independently a bond or is an optionally substituted C₁-C₄ alkylidene chain wherein up to two non-adjacent methylene units of Q are optionally replaced by CO, CO₂, CONR, OCONR, NRCO, NRCO₂, NRSO₂, SO₂NR, O, S, or NR; and each occurrence of R⁵ is independently selected from R′, halogen, NO₂, CN, OR′, SR′, N(R′)₂, NR′C(O)R′, NR′C(O)N(R′)₂, NR′CO₂R′, C(O)R′, CO₂R′, OC(O)R′, C(O)N(R′)₂, OC(O)N(R′)₂, SOR′, SO₂R′, SO₂N(R′)₂, NR′SO₂R′, NR′SO₂N(R′)₂, PO(OR′)₂, C(O)C(O)R′, or C(O)CH₂C(O)R′, and x is 0, 1, 2, or 3. In preferred embodiments, x is 1, 2, or 3.

Preferred Q—R⁵ substituents on Ar¹ for compounds of formulas I-A-i, I-A-ii, I-B-i, I-B-ii, I-C-i, I-C-ii, I-D-i, I-E-i, I-F-i, I-F-ii, II-A-i, II-A-ii, II-B-i, II-B-ii, II-C-i, II-C-ii, II-D-i, II-E-i, II-F-i, or II-F-ii are CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or two adjacent occurrences of Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur.

More preferred Q—R⁵ substituents on Ar¹ for compounds of formulas I-A-i, I-A-ii, I-B-i, I-B-ii, I-C-i, I-C-ii, I-D-i, I-E-i, I-F-i, I-F-ii, II-A-i, II-A-ii, II-B-i, II-B-ii, II-C-i, II-C-ii, II-D-i, II-E-i, II-F-i, or II-F-ii, and II-E-i are fluoro, iodo, chloro, bromo, COCH₃, CO₂CH₃, C₁₋₄alkyl (for example, methyl, ethyl, propyl, cyclopropyl, n-butyl, cyclobuyl, or t-butyl), NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₋₄alkyl), CH₂NHCO(C₁₋₄alkyl), CN, CH₂CN, OH, C₁₋₄alkoxy (for example, OCH₃, OCH₂CH₃, O(CH₂)₂CH₃, or O(CH₂)₃CH₃), optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur. Other preferred Ar¹ substituents are those substituents where two adjacent occurrences of Q—R⁵, taken together with the atoms to which they are bound, and include a fused optionally substituted saturated, partially unsaturated, or fully unsaturated 5- or 6-membered ring having 0-3 heteroatoms selected from nitrogen, oxygen, or sulfur. In more preferred embodiments, these fused substituents formed by two adjacent occurrences of Q—R⁵ include an optionally substituted group selected from methylenedioxy, ethylenedioxy, propylenedioxy, thiazolyl, oxazolyl, pyrrolyl, pyrazolyl, imidazolyl, phenyl, pyridyl, pyrimidinyl, furyl, thiophene, pyran, pyrrolidinyl, piperidinyl, piperazinyl, or morpholinyl.

In yet other embodiments, Ar¹ is phenyl and is substituted with two occurrences (x=2) of Q—R⁵ and Ar¹ is:

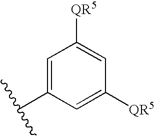

wherein each occurrence of QR⁵ is independently CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂. In certain preferred embodiments, each occurrence of QR⁵ is independently fluoro, iodo, chloro, bromo, COCH₃, CO₂CH₃, C₁₋₄alkyl (for example, methyl, ethyl, propyl, cyclopropyl, n-butyl, cyclobuyl, or t-butyl), NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₋₄alkyl), CH₂NHCO(C₁₋₄alkyl), CN, CH₂CN, OH, C₁₋₄alkoxy (for example, OCH₃, OCH₂CH₃, O(CH₂)₂CH₃, or O(CH₂)₃CH₃), optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur. In still other preferred embodiments, both occurrences of QR are methyl. In yet other preferred embodiments, at least one occurrence of QR⁵ is CF₃.

In yet other embodiments, Ar¹ is phenyl and is substituted with three occurrences (x=3) of Q—R⁵ and Ar¹ is:

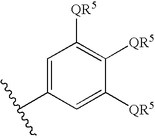

wherein each occurrence of QR⁵ is independently CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂. In certain preferred embodiments, each occurrence of QR⁵ is independently fluoro, iodo, chloro, bromo, COCH₃, CO₂CH₃, C₁₋₄alkyl (for example, methyl, ethyl, propyl, cyclopropyl, n-butyl, cyclobuyl, or t-butyl), NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₋₄alkyl), CH₂NHCO(C₁₋₄alkyl), CN, CH₂CN, OH, C₁₋₄alkoxy (for example, OCH₃, OCH₂CH₃, O(CH₂)₂CH₃, or O(CH₂)₃CH₃), optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur. In still other preferred embodiments, each occurrence of QR⁵ is independently fluoro, iodo, chloro, bromo, COCH₃, CO₂CH₃, C₁₋₄alkyl (for example, methyl, ethyl, propyl, cyclopropyl, n-butyl, cyclobuyl, or t-butyl), NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₋₄alkyl), CH₂NHCO(C₁₋₄alkyl), CN, CH₂CN, OH, optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur.

Each of the Q—R⁵ substituents described above are also optionally further substituted with one or more groups independently selected from R, OR, N(R)₂, SO₂R, halogen, NO₂, CN, SR, SO₂N(R)₂, CO₂R, C(O)R, or oxo. In more preferred embodiments, each of the Q—R⁵ groups described above are also optionally further substituted with one or two groups independently selected from methyl, ethyl, t-butyl, fluoro, chloro, bromo, oxo, CF₃, OMe, OEt, CN, SO₂Me, SO₂NH₂, NH₂, NHMe, N(Me)₂, SMe, SEt, OH, C(O)Me, NO₂, or CH₂OH.

Preferred R³ groups of for compounds of formulas I-A-i and II-A-i are those wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′. In more preferred embodiments, R³ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1. In most preferred embodiments, R³ is hydrogen or methyl.

Preferred R⁴ groups of for compounds of formulas I-A-ii and II-A-ii are those wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′. In more preferred embodiments, R⁴ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1. In still other preferred embodiments, R⁴ is hydrogen or methyl.

Preferred R³ groups of for compounds of formulas I-B-i and II-B-i are those wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′. In more preferred embodiments, R³ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1. In most preferred embodiments, R³ is hydrogen or methyl.

Preferred R⁴ groups of for compounds of formulas I-B-ii and II-B-ii are those wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′. In more preferred embodiments, R⁴ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1. In still other preferred embodiments, R⁴ is hydrogen or methyl.

Preferred R³ groups of for compounds of formulas I-C-i and II-C-i are those wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′. In more preferred embodiments, R³ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1. In most preferred embodiments, R³ is hydrogen or methyl.

Preferred R⁴ groups of for compounds of formulas I-C-ii and II-C-ii are those wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′. In more preferred embodiments, R⁴ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1. In still other preferred embodiments, R⁴ is hydrogen or methyl.

Preferred R³ groups of for compounds of formulas I-F-i and II-F-i are those wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′. In more preferred embodiments, R³ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1. In most preferred embodiments, R³ is hydrogen or methyl.

Preferred R⁴ groups of for compounds of formulas I-F-ii and II-F-ii are those wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′. In more preferred embodiments, R⁴ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1. In still other preferred embodiments, R⁴ is hydrogen or methyl.

In still other preferred embodiments, for each of the embodiments described directly above n is 0. In yet other preferred embodiments, for each of the embodiments described directly above n is 1.

Preferred R¹ and R² groups for compounds of formulas I-A-i, I-A-ii, I-B-i, I-B-ii, I-C-i, I-C-ii, I-D-i, I-E-i, I-F-i, I-F-ii, II-A-i, II-A-ii, II-B-i, II-B-ii, II-C-i, II-C-ii, II-D-i, II-E-i, II-F-i, or II-F-ii are selected from hydrogen, N(R)₂, SR, OR, or TR, or R¹ and R², taken together form an optionally substituted saturated, partially unsaturated, or fully unsaturated 5-membered ring having 0-2 heteroatoms independently selected from N, O, or S. More preferred R¹ and R² groups are hydrogen, OH, CH₃, CH₂CH₃, OCH₃, CH₂OH, CH₂OCH₃, CH₂NH₂, CH₂NHCH₃, NH₂, or CH₂NH₂, or R¹ and R², taken together, form a fused optionally substituted pyrrolyl, pyrazolyl, or imidazolyl ring. Still other preferred groups include hydrogen, NH₂, or CH₂NH₂.

In yet other preferred embodiments compounds have one of formulas II-A-i, II-B-i, II-C-i, or II-F-i wherein the compound variables are defined as:

a) x is 0, 1, 2, or 3, and Q—R⁵ is CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur;

b) R¹ and R₂ are each independently hydrogen, N(R)₂, SR, OR, or TR, or R¹ and R², taken together form an optionally substituted saturated, partially unsaturated, or fully unsaturated 5-membered ring having 0-2 heteroatoms independently selected from N, O, or S; and

c) R³ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1.

In yet other preferred embodiments compounds have one of formulas II-A-ii, II-B-ii, II-C-ii, or I-F-ii wherein one or more of the compound variables are defined as:

a) x is 0, 1, 2, or 3, and Q—R⁵ is CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur;

b) R¹ and R² are each independently hydrogen, N(R)₂, SR, OR, or TR, or R¹ and R², taken together form an optionally substituted saturated, partially unsaturated, or fully unsaturated 5-membered ring having 0-2 heteroatoms independently selected from N, O, or S; and

c) R⁴ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1.

In yet other preferred embodiments compounds have formula II-E-i, wherein one or more of the compound variables are defined as:

a) x is 0, 1, 2, or 3, and Q—R⁵ is CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur; and

b) R¹ and R² are each independently hydrogen, N(R)₂, SR, OR, or TR, or R¹ and R² taken together form an optionally substituted saturated, partially unsaturated, or fully unsaturated 5-membered ring having 0-2 heteroatoms independently selected from N, O, or S.

Exemplary compounds of Formula I (and classes and subclasses thereof) are depicted in Table 1 below:

Examples of Compounds of Formula I:

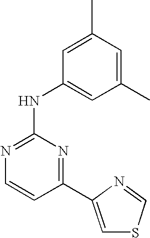

I-1

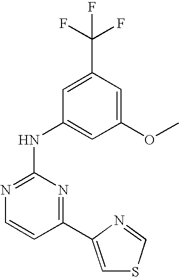

I-2

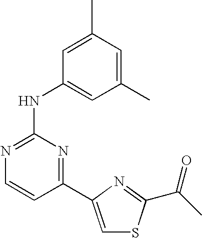

I-3

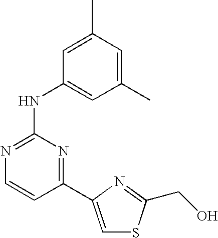

I-4

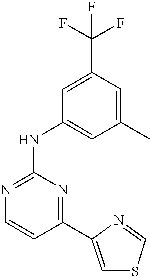

I-5

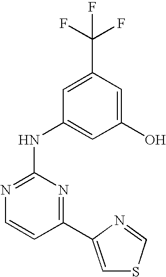

I-6

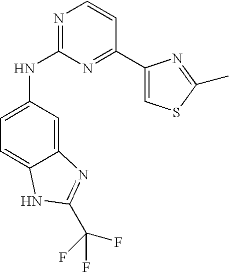

I-7

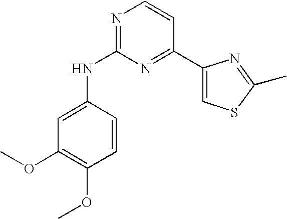

I-8

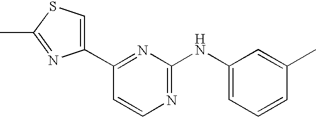

I-9

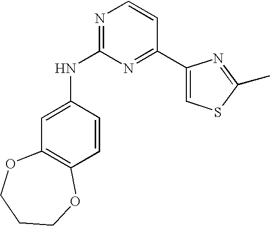

I-10

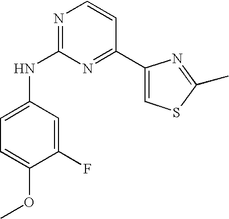

I-11

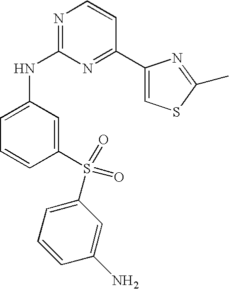

I-12

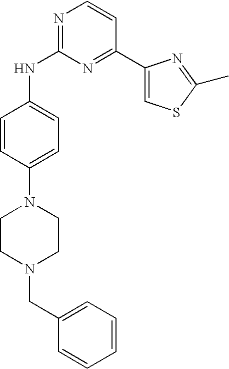

I-13

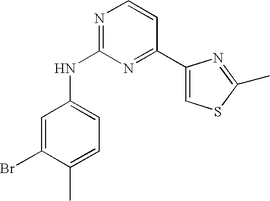

I-14

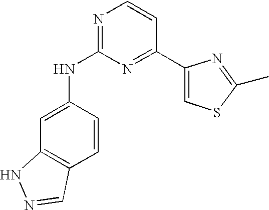

I-15

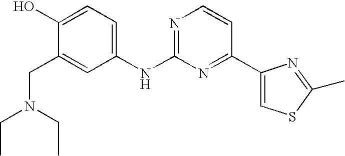

I-16

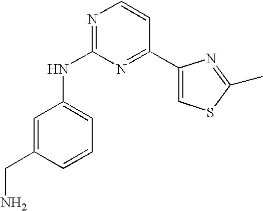

I-17

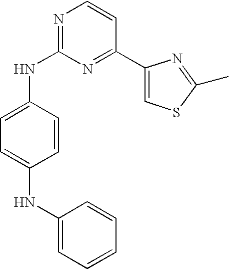

I-18

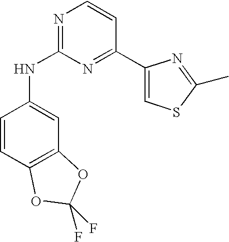

I-19

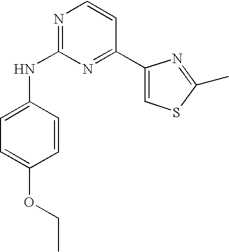

I-20

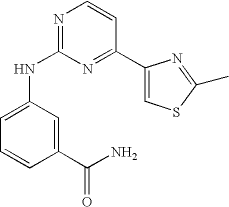

I-21

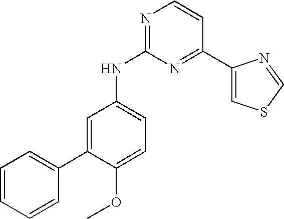

I-22

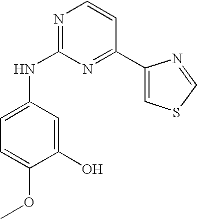

I-23

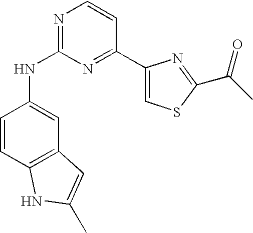

I-24

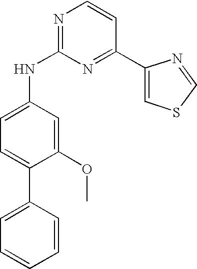

I-25

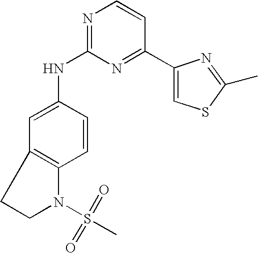

I-26

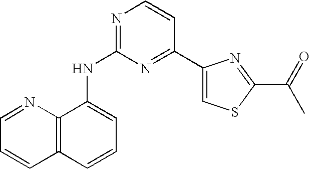

I-27

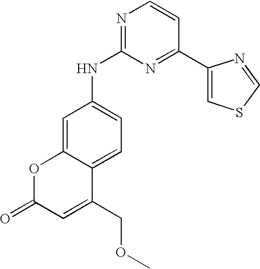

I-28

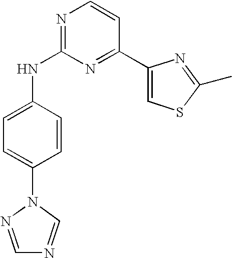

I-29

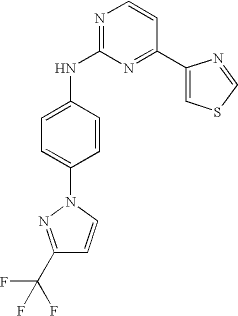

I-30

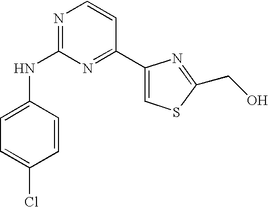

I-31

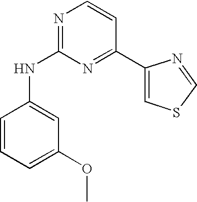

I-32

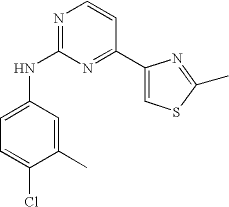

I-33

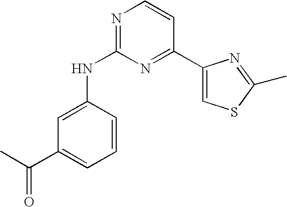

I-34

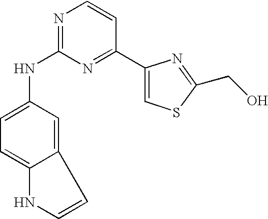

I-35

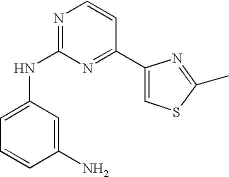

I-36

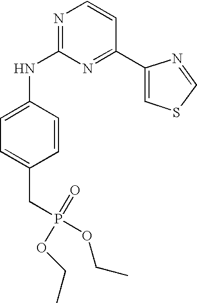

I-37

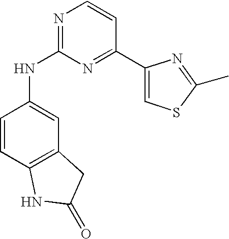

I-38

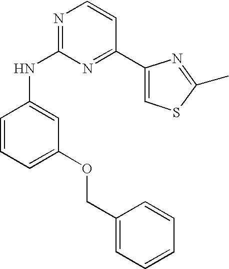

I-39

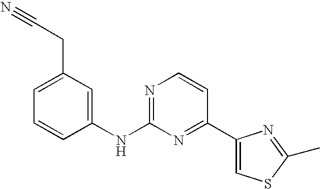

I-40

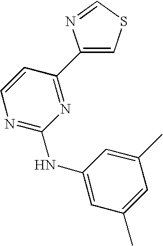

I-41

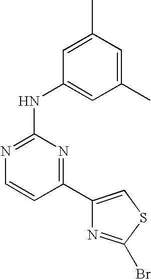

I-42

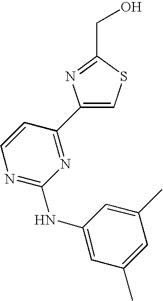

I-43

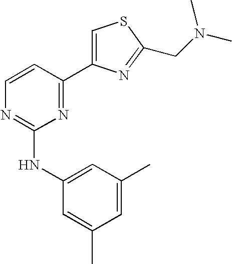

I-44

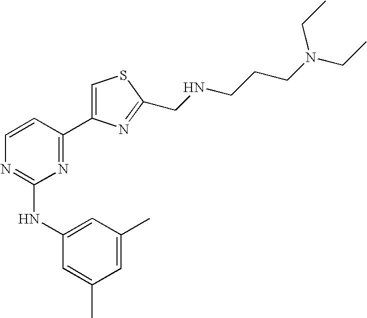

I-45

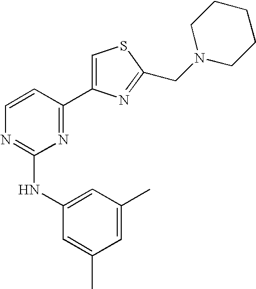

I-46

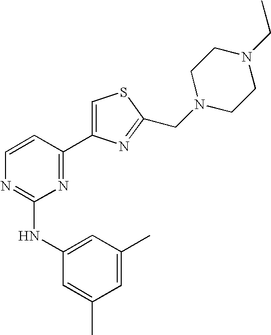

I-47

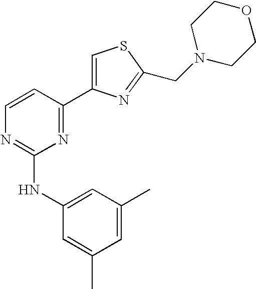

I-48

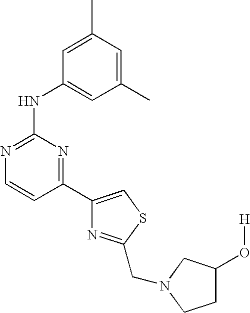

I-49

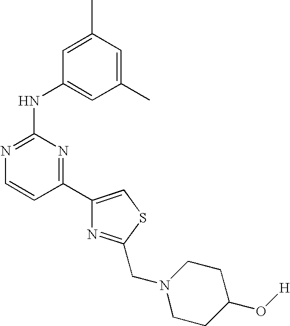

I-50

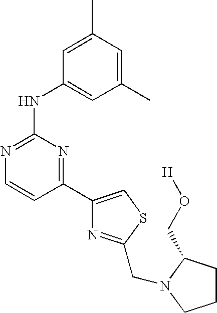

I-51

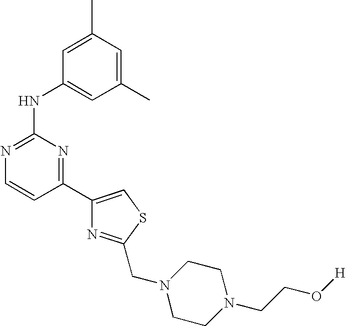

I-52

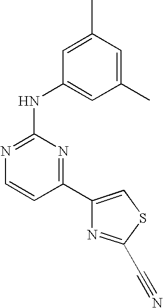

I-53

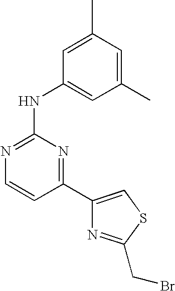

I-54

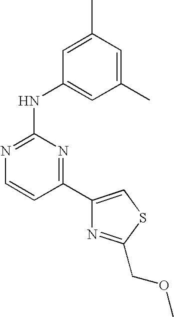

I-55

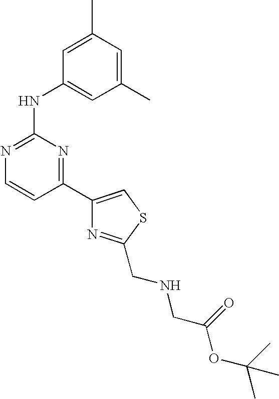

I-56

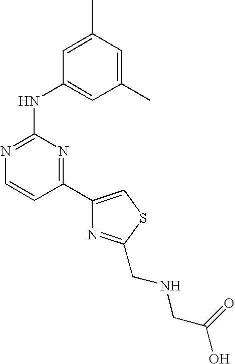

I-57

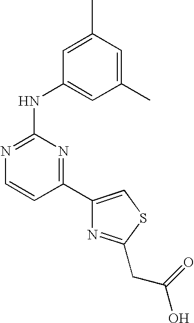

I-58

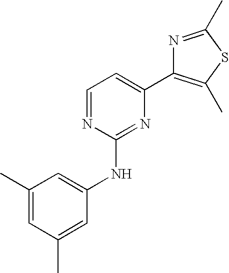

I-59

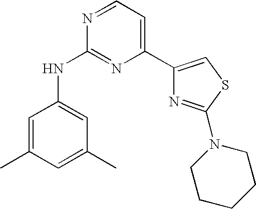

I-60

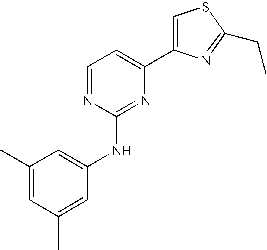

I-61

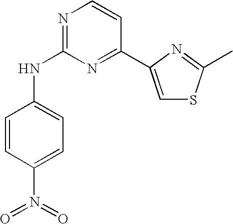

I-62

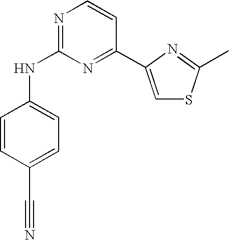

I-63

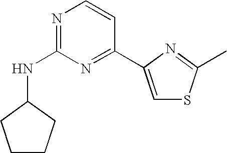

I-64

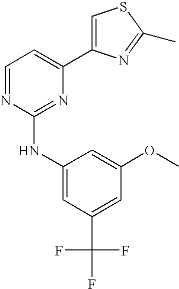

I-65

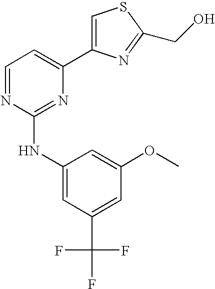

I-66

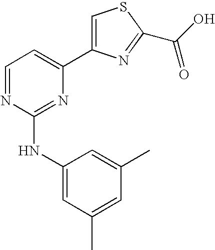

I-67

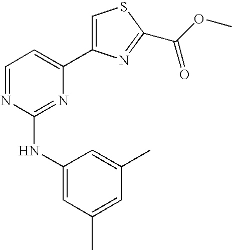

I-68

4. General Synthetic Methodology:

The compounds of this invention may be prepared in general by methods known to those skilled in the art for analogous compounds, as illustrated by the general scheme below, and the preparative examples that follow.

Scheme I below shows a general synthetic route that may be used used for preparing certain compounds of the invention.

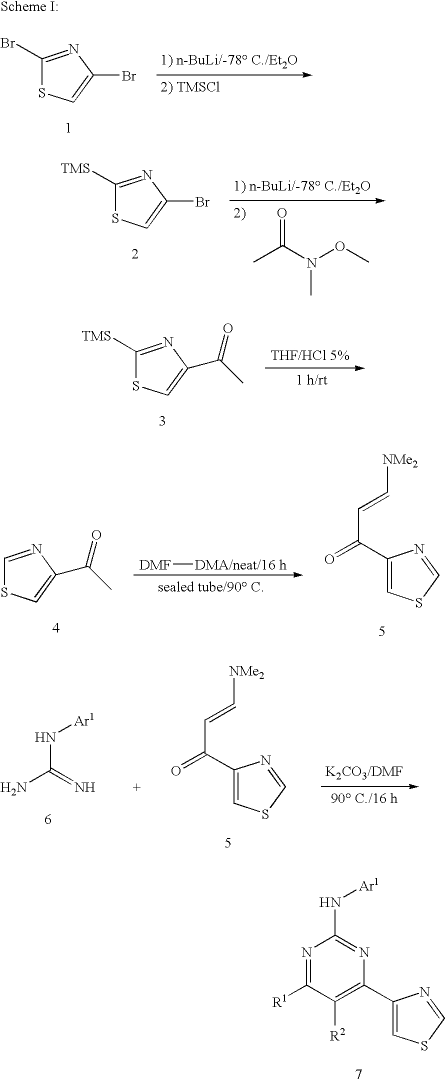

As depicted above, after preparation of 3-acetyl thiazole (4), a solution of (4) THF is treated with dimethylformamide-dimethylacetal and the resulting mixture stirred at room temperature over night. The reaction mixture is concentrated in vacuo and the concentrate triturated with diethyl ether to afford 5.

To prepare intermediate 6, a mixture of Ar¹NH₂ and cyanamide in HCl (4N in dioxane) is heated at 120° C. overnight. After cooling to room temperature, aqueous work-up affords the desired guanidine compound 6. One of skill in the art would recognize that a wide variety of aryl guanidines may be prepared and may thus be used to prepare compounds of formula I with a wide variety of Ar¹ rings.

In step (b), guanidine 6 is combined with enaminone 5 in DMF in a sealed tube. The resulting mixture is heated at reflux overnight then concentrated and the crude product purified by column chromatography to afford the desired pyrimidine compound 7. The details of the conditions used for producing these compounds are set forth in the Examples.

In one exemplary embodiment, as shown in Scheme II, phenylguanidine 6a is prepared and used to generate compounds of general formula 7a.

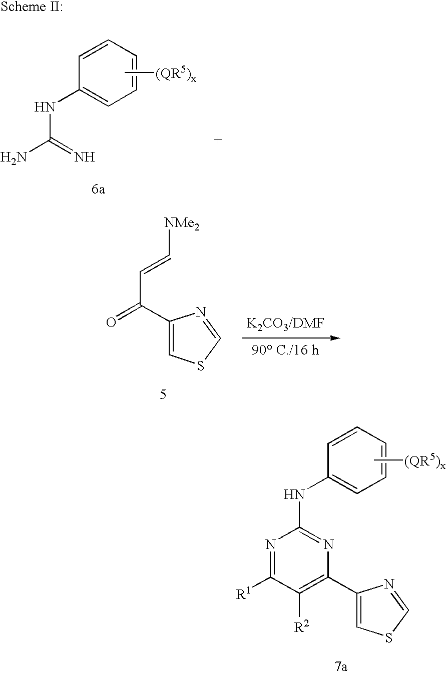

Schemes III, IV, and V, below depict the synthesis of certain exemplary compounds of the invention.

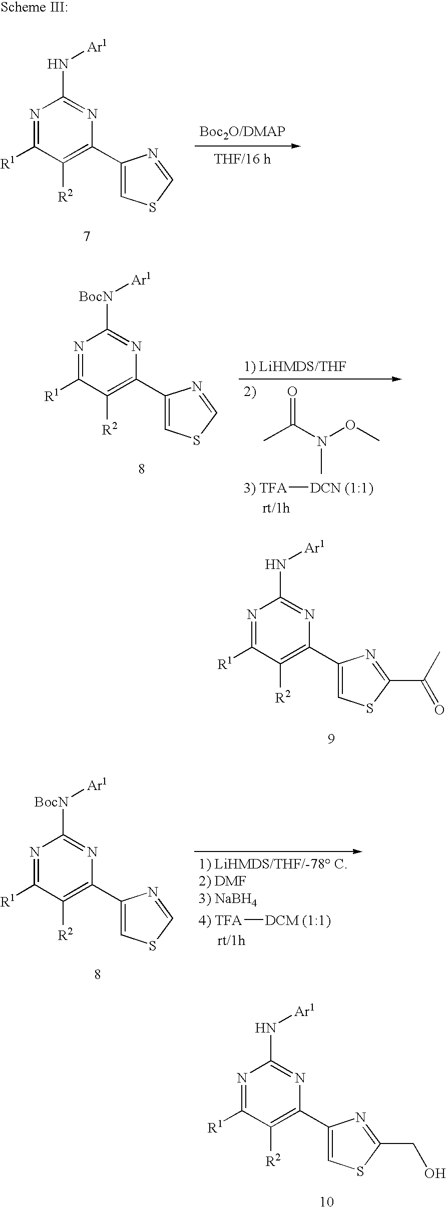

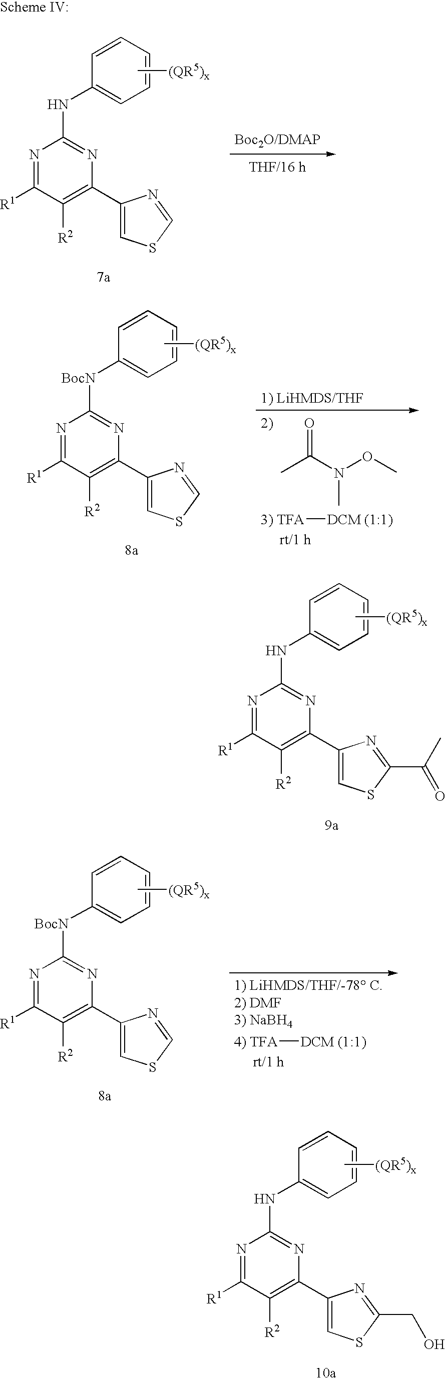

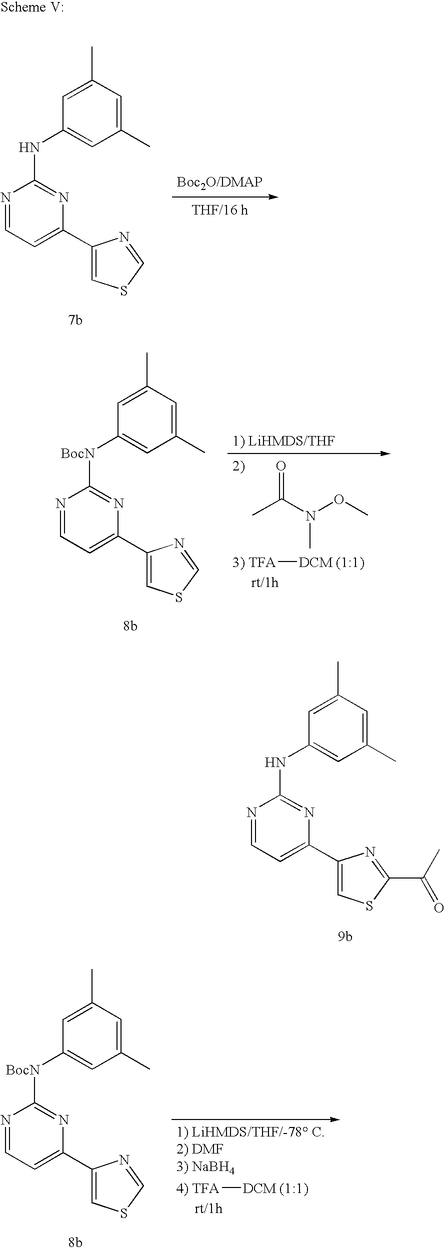

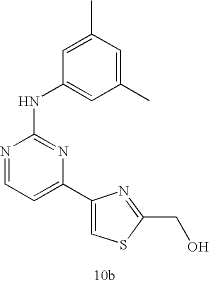

Although certain exemplary embodiments are depicted and described above and herein, it will be appreciated that additional compounds of the invention can be prepared according to the methods described generally above using appropriate starting materials by methods generally available to one of ordinary skill in the art.

5. Uses, Formulation and Administration

Pharmaceutically Acceptable Compositions

As discussed above, the present invention provides compounds that are inhibitors of protein kinases, and thus the present compounds are useful for the treatment of diseases, disorders, and conditions including, but not limited to immunodeficiency disorders, inflammatory diseases, allergic diseases, autoimmune diseases, proliferative disorders, immunologically-mediated diseases, or respiratory disorders. Accordingly, in another aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof. As used herein, the term “inhibitorily active metabolite or residue thereof” means that a metabolite or residue thereof is also an inhibitor of SYK or ZAP-70 kinase.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Uses of Compounds and Pharmaceutically Acceptable Compositions

In yet another aspect, a method for the treatment or lessening the severity of immunodeficiency disorders, inflammatory diseases, allergic diseases, autoimmune diseases, proliferative disorders, immunologically-mediated diseases, or respiratory disorders is provided comprising administering an effective amount of a compound, or a pharmaceutically acceptable composition comprising a compound to a subject in need thereof. In certain embodiments of the present invention an “effective amount” of the compound or pharmaceutically acceptable composition is that amount effective for treating or lessening the severity of immunodeficiency disorders, inflammatory diseases, allergic diseases, autoimmune diseases, proliferative disorders, immunologically-mediated diseases, or respiratory disorders. The compounds and compositions, according to the method of the present invention, may be administered using any amount and any route of administration effective for treating or lessening the severity of immunodeficiency disorders, inflammatory diseases, allergic diseases, autoimmune diseases, proliferative disorders, immunologically-mediated diseases, or respiratory disorders. The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of protein kinases. In one embodiment, the compounds and compositions of the invention are inhibitors of one or more of SYK or ZAP-70, and thus, without wishing to be bound by any particular theory, the compounds and compositions are particularly useful for treating or lessening the severity of a disease, condition, or disorder where activation of one or more of SYK or ZAP-70 is implicated in the disease, condition, or disorder. When activation of SYK or ZAP-70 is implicated in a particular disease, condition, or disorder, the disease, condition, or disorder may also be referred to as “SYK or ZAP-70-mediated disease” or disease symptom. Accordingly, in another aspect, the present invention provides a method for treating or lessening the severity of a disease, condition, or disorder where activation or one or more of SYK or ZAP-70 is implicated in the disease state.

The activity of a compound utilized in this invention as an inhibitor of SYK or ZAP-70, may be assayed in vitro, in vivo or in a cell line. In vitro assays include assays that determine inhibition of either the phosphorylation activity or ATPase activity of activated SYK or ZAP-70. Alternate in vitro assays quantitate the ability of the inhibitor to bind to SYK or ZAP-70. Inhibitor binding may be measured by radiolabelling the inhibitor prior to binding, isolating the inhibitor/SYK or inhibitor/ZAP-70, complex and determining the amount of radiolabel bound. Alternatively, inhibitor binding may be determined by running a competition experiment where new inhibitors are incubated with SYK or ZAP-70 bound to known radioligands.

The term “measurably inhibit”, as used herein means a measurable change in SYK or ZAP-70 activity between a sample comprising said composition and a SYK or ZAP-70 kinase and an equivalent sample comprising SYK or ZAP-70 kinase in the absence of said composition.

The term “SYK-mediated disease” or “SYK-mediated condition”, as used herein, means any disease or other deleterious condition in which SYK protein kinase is known to play a role. Such conditions include, without limitation, allergic disorders, especially asthma.

The term “ZAP-70-mediated condition”, as used herein means any disease or other deleterious condition in which ZAP-70 is known to play a role. Such conditions include, without limitation, autoimmune, inflammatory, proliferative, and hyperproliferative diseases and immunologically-mediated diseases including rejection of transplanted organs or tissues and Acquired Immunodeficiency Syndrome (AIDS).

For example, ZAP-70-mediated conditions include diseases of the respiratory tract including, without limitation, reversible obstructive airways diseases including asthma, such as bronchial, allergic, intrinsic, extrinsic and dust asthma, particularly chronic or inveterate asthma (e.g. late asthma airways hyper-responsiveness) and bronchitis. Additionally, ZAP-70 diseases include, without limitation, those conditions characterised by inflammation of the nasal mucus membrane, including acute rhinitis, allergic, atrophic thinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis, seasonal rhinitis including rhinitis nervosa (hay fever) and vasomotor rhinitis, sarcoidosis, farmer's lung and related diseases, fibroid lung and idiopathic interstitial pneumonia.

ZAP-70-mediated conditions also include diseases of the bone and joints including, without limitation, (pannus formation in) rheumatoid arthritis, seronegative spondyloarthropathis (including ankylosing spondylitis, psoriatic arthritis and Reiter's disease), Behcet's disease, Sjogren's syndrome, and systemic sclerosis.

ZAP-70-mediated conditions also include diseases and disorders of the skin, including, without limiation, psoriasis, systemic sclerosis, atopical dermatitis, contact dermatitis and other eczematous dermatitis, seborrhoetic dermatitis, Lichen planus, Pemphigus, bullous Pemphigus, epidermolysis bullosa, urticaria, angiodermas, vasculitides, erythemas, cutaneous eosinophilias, uveitis, Alopecia, areata and vernal conjunctivitis.

ZAP-70-mediated conditions also include diseases and disorders of the gastrointestinal tract, including, without limitation, Coeliac disease, proctitis, eosinophilic gastro-enteritis, mastocytosis, pancreatitis, Crohn's disease, ulcerative colitis, food-related allergies which have effects remote from the gut, e.g. migraine, rhinitis and eczema.

ZAP-70-mediated conditions also include those diseases and disorders of other tissues and systemic disease, including, without limiation, multiple sclerosis, artherosclerosis, acquired immunodeficiency syndrome (AIDS), lupus erythematosus, systemic lupus, erythematosus, Hashimoto's thyroiditis, myasthenia gravis, type I diabetes, nephrotic syndrome, eosinophilia fascitis, hyper IgE syndrome, lepromatous leprosy, sezary syndrome and idiopathic thrombocytopenia purpura, restenosis following angioplasty, tumours (for example leukemia, lymphomas), artherosclerosis, and systemic lupus erythematosus.

ZAP-70-mediated conditions also include allograft rejection including, without limitation, acute and chronic allograft rejection following for example transplantation of kidney, heart, liver, lung, bone marrow, skin and cornea; and chronic graft versus host disease.

It will also be appreciated that the compounds and pharmaceutically acceptable compositions of the present invention can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention or pharmaceutically acceptable compositions thereof may also be incorporated into compositions for coating implantable medical devices, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device.

Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

Another aspect of the invention relates to inhibiting SYK or ZAP-70 activity in a biological sample or a patient, which method comprises administering to the patient, or contacting said biological sample with a compound of formula I or a composition comprising said compound. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of SYK or ZAP-70 kinase activity in a biological sample is useful for a variety of purposes that are known to one of skill in the art. Examples of such purposes include, but are not limited to, blood transfusion, organ-transplantation, biological specimen storage, and biological assays.

### EXAMPLES

### A) Synthesis of Exemplary Compounds of the Invention:

### Example 1

### (Referring to compounds as numbered in Schemes I and II) Synthesis of 7a:

A) 4-Bromo-2-(trimethyl-silanyl)-thiazole (2).

To a stirred solution of n-BuLi (2M in pentane; 12.35 mL, 0.0247 mol) in 70 mL of dry ether at −78° C. was added dropwise 2,4-dibromothiazole in 30 mL of ether over a period of 30 minutes. The mixture was stirred for 1 h, then TMSCl (2.87 mL, 0.0226 mol) was added dropwise over 10 minutes. After lh of stirring at −78° C. the reaction mixture was washed with sat'd NaHCO₃ and extracted with ether. The organic layer was dried with Na₂SO₄ and concentrated in vacuo to give 4.0 g (82%) of 2 as an oil that was used directly for the next step.

B) 1-[2-(Trimethyl-silanyl)-thiazol-4-yl]-ethanone (3).

To a stirred solution of n-BuLi (2M in pentane; 12.7 mL, 0.025 mol) in 70 mL of dry ether at −78° C. was added dropwise 16 (4.0 g, 0.017 mol) in 30 mL of ether over a period of 30 minutes. The mixture was stirred for lh, then N-methoxy-N-methylacetamide (2.16 mL, 0.02 mol) was added dropwise over 10 minutes. After 1 h of stirring at −78° C. the reaction mixture was washed with sat'd NaHCO₃ and extracted with ether. The organic layer was dried with Na₂SO₄ and concentrated in vacuo to give 2.4 g (71%) of 3 as an oil that was used directly for the next step.

C) 1-Thiazol-4-yl-ethanone (4).

A mixture of 17 (2.4 g, 12.4 mmol) and 4 mL of 5% HCl in 40 mL of THF were stirred at rt for 1 h. The mixture was diluted with ether and washed with sat'd NaHCO₃ and dried with Na₂SO₄ and concentrated in vacuo to give 1.44 g (94%) of 4 as an oil that was used directly for the next step.

D) 3-Dimethylamino-1-thiazol-4-yl-propenone (5).

A solution of 4 (2.48g, 0.124 mol) in 7.5 mL of DMF-DMA was heated at 90° C. in a sealed tube for 16 h. The precipitate that formed upon cooling was collected to give 0.5 g (25%) of the desired enaminone 5. Reaction of enaminone 5 and 6a under the general conditions described above and herein yields desired compounds 7a.

### Example 2

### (Referring to compound numbering in Scheme V): Synthesis of 9b:

A) (3,5-Dimethyl-phenyl)-(4-thiazol-4-yl-pyrimidin-2-yl)-carbamic acid tert-butyl ester (8b).

To a solution of 7b (462 mg, 0.00164 mol) in 10 niL of anhydrous THF at rt was added boc anhydride (446 mg, 0.00204) followed by a catalytic amount of DMAP. The reaction mixture was stirred for 16 h. Concentrated to dryness and passed through a short plug of silica (30% ethyl acetate/70% hexanes) to give 631 mg (100%) of the desired product 8b.

B) 1-{4-[2-(3,5-Dimethyl-phenylamino)-pyrimidin-4-yl]-thiazol-2-yl }-ethanone (9b).

To a stirred solution of LiHMDS (1M in THF; 0.313 mL, 0.314 mmol) in 2 rnL of dry THF at −78° C. was added dropwise 8b (100 mg, 0.261 mmol) in 1 mL of ether over a period of 3 minutes. The mixture was stirred for 1 h at −78° C. then warmed up to rt and stirred for 15 minutes. Cooled to −78° C., then N-methoxy-N-methylacetamide (0.033 mL, 0.314 mmol) was added dropwise over 5 minutes. After 1 h of stirring at −78° C. the reaction mixture was washed with sat'd NaHCO₃ and extracted with ether. The organic layer was dried with Na₂SO₄ and concentrated in vacuo to give a crude solid residue that was subjected to flash chromatography (40% ethyl acetate/60% hexanes) to give 49 mg (44%) of a carbamate as a solid. Removal of the t-butoxycarbonyl group with TFA-DCM (1:1) at rt for 1 h followed by flash chromatography (40% ethyl acetate/60% hexanes) gave 20 mg (53%) of the desired product 9b.

### Example 3

### (Referring to compound numbering in Scheme V): Synthesis of {4-[2-(3,5-Dimethyl-phenylamino)-pyrimidin-4-yl]-thiazol-2-yl}-methanol (10b).

To a stirred solution of LiHMDS (1M in THF; 0.313 mL, 0.314 mmol) in 2 mL of dry THF at −78° C. was added dropwise 8b (100 mg, 0.261 mmol) in 1 mL of ether over a period of 3 minutes. The mixture was stirred for 1 h at −78° C. then warmed up to rt and stirred for 15 minutes. Cooled to −78° C., then DMF (0.024 mL, 0.314 mmol) was added dropwise over 5 minutes. After 1 h of stirring at −78° C. the reaction mixture was washed with sat'd NaHCO₃ and extracted with ether. The organic layer was dried with Na₂SO₄ and concentrated in vacuo to give a crude solid residue that was subjected to sodium borohydride (15 mg, 1.5 eq.) reduction in 2 mL of methanol. Quenched with 0.5 mL of 1M HCl and extracted with ethyl acetate. The organic layer was dried with Na₂SO₄ and concentrated in vacuo to give a crude solid residue that was deprotected as above. Flash chromatography (40% ethyl acetate/60% hexanes) gave 29 mg (20%) of the desired product 10b.

B) Biological Data:

### Example 1

### SYK Inhibition Assay:

Compounds were screened for their ability to inhibit SYK using a standard coupled enzyme assay (Fox et al., Protein Sci. 1998, 7, 2249). Reactions were carried out in 100 mM HEPES (pH 7.5), 10 mM MgCl₂, 25 mM NaCl, 1 mM DTT and 1.5% DMSO. Final substrate concentrations in the assay were 200 μM ATP (Sigma chemical Co.) and 4 μM poly Gly—Tyr peptide (Sigma Chemical Co.). Assays were carried out at 30° C. and 200 nM SYK. Final concentrations of the components of the coupled enzyme system were 2.5 mM phosphoenolpyruvate, 300 μM NADH, 30 μg/ml pyruvate kinase and 10 μg/ml lactate dehydrogenase.

An assay stock buffer solution was prepared containing all of the reagents listed above, with the exception of SYK, DTT, and the test compound of interest of the present invention. 56 μl of the test reaction was placed in a 96 well plate followed by the addition of 1 μl of 2 mM DMSO stock containing the test compound of the present invnetion (final compound concentration 30 μM). The plate was pre-incubated for ˜10 minutes at 30° C. and the reaction initiated by the addition of 10 μl of enzyme (final concentration 25 nM). Rates of reaction were obtained using a BioRad Ultramark plate reader (Hercules, Calif.) over a 5 minute read time at 30° C., and Kᵢ values for the compounds of the present invention were determined according to standard methods.

Compounds of the invention are useful as inhibitors of SYK. The following compounds exhibit Kᵢ values of 5.0 μM or less: I-1, 1-2, 1-3, 1-4, 1-5, and 1-6.

### Example 2

### ZAP-70 Inhibition Assay

Compounds were screened for their ability to inhibit ZAP-70 using a standard coupled enzyme assay (Fox et al., Protein Sci. 1998, 7, 2249). Assays were carried out in a mixture of 100 mM HEPES (pH 7.5), 10 mM MgCl₂, 25 mM NaCl, 2 mM DTT and 3% DMSO. Final substrate concentrations in the assay were 100 μM ATP (Sigma Chemicals) and 20 μM peptide (poly-4EY, Sigma Chemicals). Assays were carried out at 30° C. and 60 nM ZAP-70. Final concentrations of the components of the coupled enzyme system were 2.5 mM phosphoenolpyruvate, 300 μM NADH, 30 μg/ml pyruvate kinase and 10 μg/ml lactate dehydrogenase.

An assay stock buffer solution was prepared containing all of the reagents listed above, with the exception of ZAP-70 and the test compound of interest of the present invention. 55 μl of the stock solution was placed in a 96 well plate followed by addition of 2 μl of DMSO stock containing serial dilutions of the test compound of the present invention (typically starting from a final concentration of 15 μM). The plate was preincubated for 10 minutes at 30° C. and the reaction initiated by addition of 10 μl of enzyme (final concentration 60 nM). Initial reaction rates were determined with a Molecular Devices SpectraMax Plus plate reader over a 15 minute time course. Kᵢ data was calculated from non-linear regression analysis using the Prism software package (GraphPad Prism version 3.Oa for Macintosh, GraphPad Software, San Diego Calif., USA).

Compounds of the invention are useful as inhibitors of ZAP-70. The following compounds exhibit Kᵢ values of 5.0 μM or less I-1, 1-2, 1-3, 1-4, 1-5, and 1-6.

While a number of embodiments of this invention have been described, it is apparent that the basic examples described herein may be altered to provide other embodiments that utilize the compounds and methods of this invention. Therefore, it will be appreciated that the scope of this invention is to be defined by the appended claims rather than by the specific embodiments that have been represented by way of example.

1. A compound of formula (I): 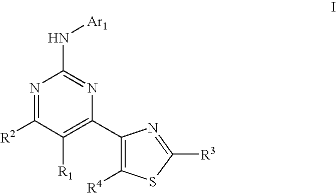
or a pharmaceutically acceptable salt thereof, wherein:
R¹ and R² are each independently R, halogen, CN, NO₂, or TR, or R¹ and R² taken together form an optionally substituted saturated, partially unsaturated, or fully unsaturated 5- or 6-membered ring having 0-3 heteroatoms independently selected from N, O, or S;
T is an optionally substituted C₁-C₄ alkylidene chain wherein up to two methylene units of T are optionally and independently replaced by O, N(R), C(O), S, SO, or SO₂;
Ar¹ is an optionally substituted ring selected from: an aryl group selected from a 5-6 membered monocyclic or an 8-10 membered bicyclic ring having 0-5 heteroatoms independently selected from nitrogen, oxygen, or sulfur; a 3-8-membered saturated or partially unsaturated monocyclic ring having 0-3 heteroatoms independently selected from nitrogen, oxygen, or sulfur; or an 8-10-membered saturated or partially unsaturated bicyclic ring system having 0-5 heteroatoms independently selected from nitrogen, oxygen, or sulfur; wherein Ar¹ is optionally substituted at one or more carbon atoms with 0-5 occurrences of —Q—R⁵, and at one or more substitutable nitrogen atoms with —R⁶ and each occurrence of R6 is independently R′, —COR′, —CO₂(C₁₋₆ aliphatic), —CON(R′)₂, —SO₂N(R′)₂, or —SO₂R′;
R³ and R⁴ are each independently Z—R⁷;
each occurrence of Q and Z is independently a bond or an optionally substituted C₁-C₆ alkylidene chain wherein up to two non-adjacent methylene units of Q are optionally replaced by CO, CO₂, COCO, CONR, OCONR, NRNR, NRNRCO, NRCO, NRCO₂, NRCONR, SO, SO₂, NRSO₂, SO₂NR, NRSO₂NR, O, S, or NR;
each occurrence of R⁵ and R⁷ is independently R′, halogen, NO₂, CN, OR′, SR′, N(R′)₂, NR′C(O)R′, NR′C(O)N(R′)₂, NR′CO₂R′, C(O)R′, CO₂R′, OC(O)R′, C(O)N(R′)₂, OC(O)N(R′)₂, SOR′, SO₂R′, SO₂N(R′)₂, NR′SO₂R′, NR′SO₂N(R′)₂, PO(OR′)₂, C(O)C(O)R′, or C(O)CH₂C(O)R′; and
each occurrence of R is independently hydrogen or an optionally substituted C₁₋₆ aliphatic group; and each occurrence of R′ is independently hydrogen or an optionally substituted group selected from C₁₋₈ aliphatic, C₆₋₁₀ aryl, a heteroaryl ring having 5-10 ring atoms, or a heterocyclyl ring having 3-10 ring atoms, or wherein two occurrences of R taken together, R and R′ taken together, or two occurrences of R′ taken together, form an optionally substituted saturated, partially unsaturated, or fully unsaturated 3-8 membered ring having 0-3 heteroatoms independently selected from nitrogen, oxygen, or sulfur;
provided that:
when R¹ and R² are both hydrogen, R³is hydrogen, R⁴ is CN, or
when R¹ and R² are both hydrogen, R³is NH₂, R⁴ is CN, then Ar¹ is not phenyl or pyridyl substituted with one or two occurrences of Cl, Me, CH₂NRR′, C(O)NRR′, or SO₂NRR′, wherein R and R′ taken together form an optionally substituted saturated 6- or 7-membered ring having 1 or 2 heteroatoms independently selected from nitrogen or oxygen.

2. The compound of claim 1, wherein Ar¹ are optionally substituted rings selected from:
(a) a phenyl, indanyl, or naphthyl ring;
(b) a 5-6 membered heterocyclic ring having 1-3 heteroatoms independently selected from nitrogen, oxygen, or sulfur; or
(c) a 5-6 membered monocyclic or 9-10 membered bicyclic heteroaryl ring having 1-3 heteroatoms independently selected from oxygen, nitrogen, or sulfur.

3. The compound of claim 1, wherein Ar¹ are optionally substituted rings selected from:
(a) a phenyl ring;
(b) a 5-6 membered heterocyclic ring having 1-3 heteroatoms independently selected from nitrogen, oxygen, or sulfur; or
(c) a 5-6 membered monocyclic heteroaryl ring having 1-3 heteroatoms independently selected from oxygen, nitrogen, or sulfur.

4. The compound of claim 1, wherein Ar¹ is selected from any one of a-bb: 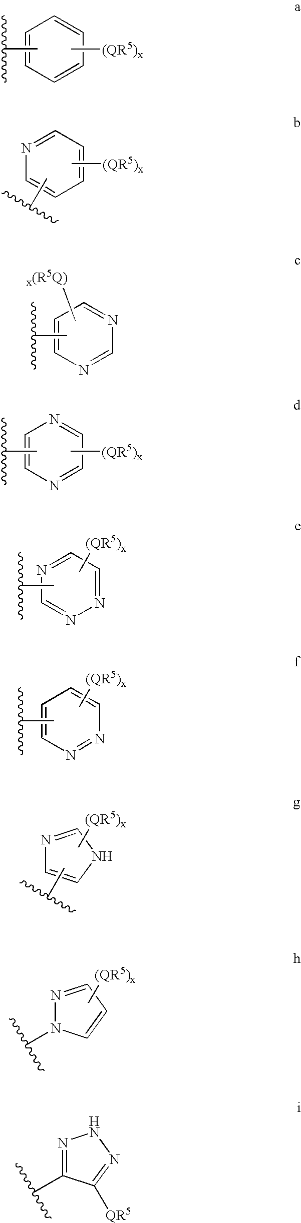 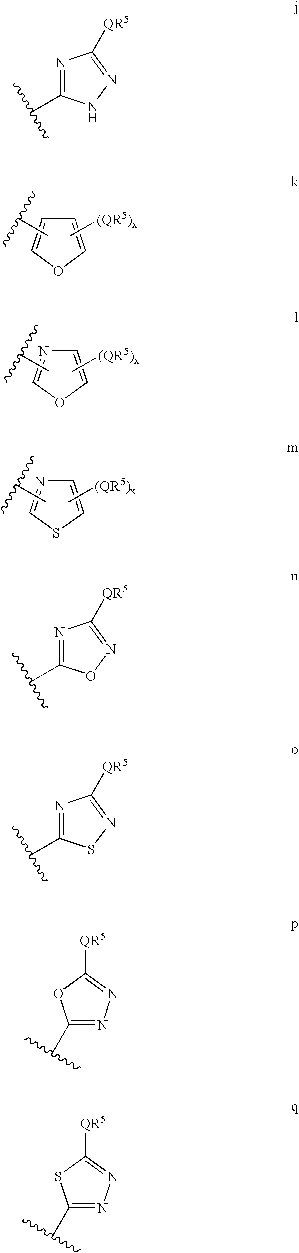 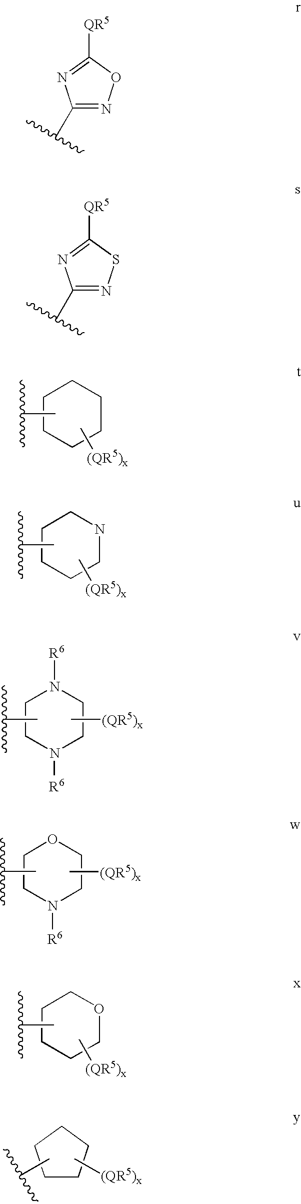 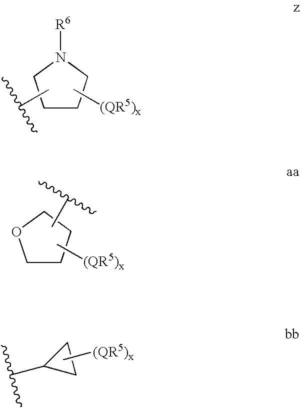
wherein x is 0-5.

5. The compound of claim 1, wherein Ar¹ is optionally substituted phenyl, pyrimidinyl, or pyridyl.

6. The compound of claim 1, wherein Ar¹ is phenyl and is substituted with two (x=2) or three (x=3) occurrences of Q—R⁵ and Ar¹ is: 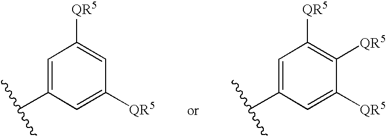
wherein each occurrence of QR⁵ is independently CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂.

7. The compound of claim 1, wherein Q is independently a bond or is an optionally substituted C₁-C₄ alkylidene chain wherein up to two non-adjacent methylene units of Q are optionally replaced by CO, CO₂, CONR, OCONR, NRCO, NRCO₂, NRSO₂, SO₂NR, O, S, or NR; and each occurrence of R⁵ is independently selected from R′, halogen, NO₂, CN, OR′, SR′, N(R′)₂, NR′C(O)R′, NR′C(O)N(R′)₂, NR′CO₂R′, C(O)R′, CO₂R′, OC(O)R′, C(O)N(R′)₂, OC(O)N(R′)₂, SOR′, SO₂R′, SO₂N(R′)₂, NR′SO₂R′, NR′SO₂N(R′)₂, PO(OR′)₂, C(O)C(O)R′, or C(O)CH₂C(O)R′, and x is 0, 1, 2, or 3.

8. The compound of claim 1, wherein Q—R⁵ substituents on Ar¹ are CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or two adjacent occurrences of Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur.

9. The compound of claim 1, wherein Q—R⁵ substituents on Ar¹ are fluoro, iodo, chloro, bromo, COCH₃, CO₂CH₃, C₁₋₄alkyl, NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₋₄alkyl), CH₂NHCO(C₁₋₄alkyl), CN, CH₂CN, OH, C₁₋₄alkoxy, optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur.

10. The compound of claim 1, wherein R¹ and R² groups of formula I are each independently hydrogen, N(R)₂, SR, OR, or TR, or R¹ and R², taken together form an optionally substituted saturated, partially unsaturated, or fully unsaturated 5-membered ring having 0-2 heteroatoms independently selected from N, O, or S.

11. The compound of claim 1, wherein R¹ and R² groups are each independently hydrogen, OH, CH₃, CH₂CH₃, OCH₃, CH₂OH, CH₂OCH₃, CH₂NH₂, CH₂NHCH₃, NH₂, or CH₂NH₂, or R¹ and R², taken together, form a fused optionally substituted pyrrolyl, pyrazolyl, or imidazolyl ring.

12. The compound of claim 1, wherein R³ and R⁴are each independently Z—R⁷ wherein Z is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′, or wherein R³ and R⁴, taken together form an optionally substituted saturated, partially unsaturated, or fully unsaturated 5- or 6-membered ring having 0-3 heteroatoms independently selected from nitrogen, oxygen, or sulfur.

13. The compound of claim 1, wherein R³ and R⁴ are each independently hydrogen, CN, halogen, OH, SH, NH₂, CO₂H, COH, CONH₂, SO₂NH₂, NO₂, (CH₂)ₙNRR⁷, wherein R and R⁷, taken together with the nitrogen atom to which they are bound form an optionally substituted 3-8-membered saturated or partially unsaturated ring having 1-3 heteroatoms selected from nitrogen, oxygen, or sulfur, or R³ and R⁴, taken together with the atoms to which they are bound, form an optionally substituted saturated, partially unsaturated, or fully unsaturated 5- or 6-membered ring having 0-3 heteroatoms independently selected from nitrogen, oxygen, or sulfur, and n is 0, 1, 2, 3, 4, or 5.

14. The compound of claim 1, wherein one of R³ or R⁴ is hydrogen, and the other of R³ or R⁴ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is hydrogen, (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur.

15. The compound of claim 14, wherein R³is hydrogen.

16. The compound of claim 14, wherein R⁴is hydrogen.

17. The compound of claim 1, having one of formulas I-A-i, I-A-ii, I-B-i, I-B-ii, I-C-i, I-C-ii, I-D-i, or I-E-i:

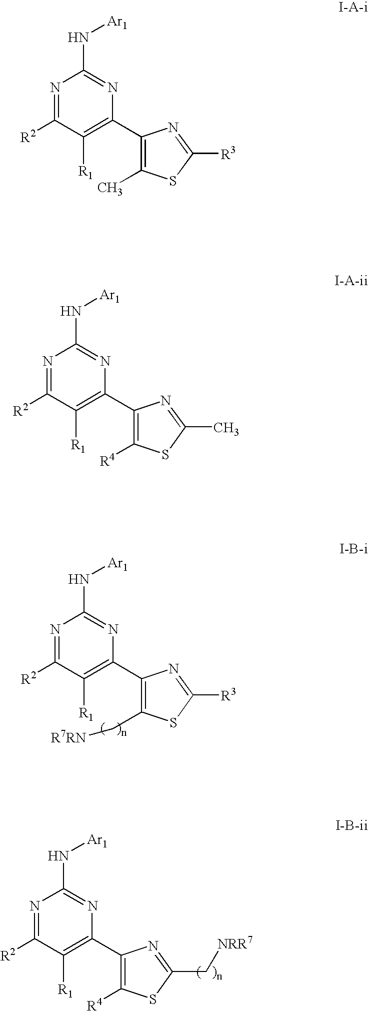

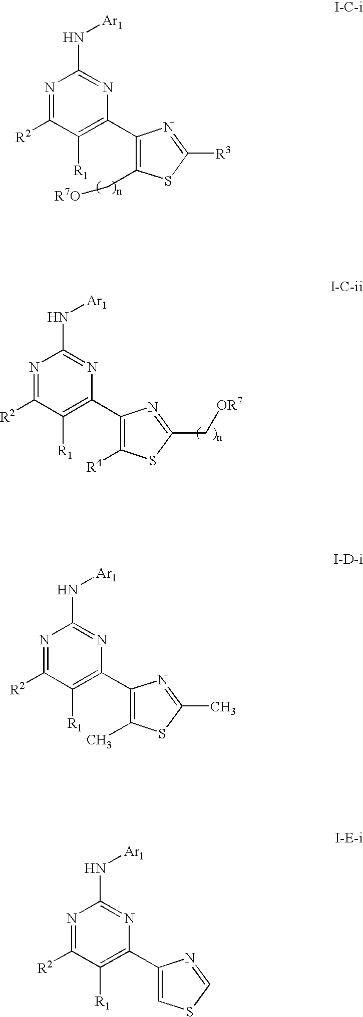

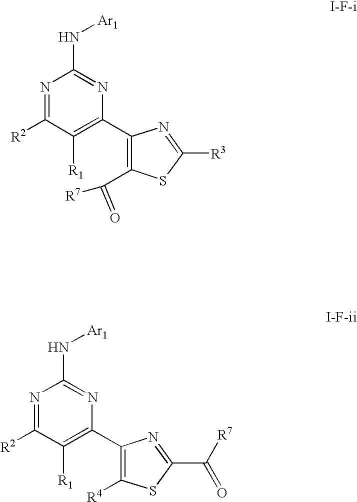

18. The compound of claim 17, wherein Ar¹ is:
(a) a phenyl, indanyl, or naphthyl ring;
(b) a 5-6 membered heterocyclic ring having 1-3 heteroatoms independently selected from nitrogen, oxygen, or sulfur; or
(c) a 5-6 membered monocyclic or 9-10 membered bicyclic heteroaryl ring having 1-3 heteroatoms independently selected from oxygen, nitrogen, or sulfur.

19. The compound of claim 17, wherein Ar¹ is:
(a) a phenyl ring;
(b) a 5-6 membered heterocyclic ring having 1-3 heteroatoms independently selected from nitrogen, oxygen, or sulfur; or
(c) a 5-6 membered monocyclic heteroaryl ring having 1-3 heteroatoms independently selected from oxygen, nitrogen, or sulfur.

20. The compound of claim 17, wherein Ar¹ is any one of a-bb: 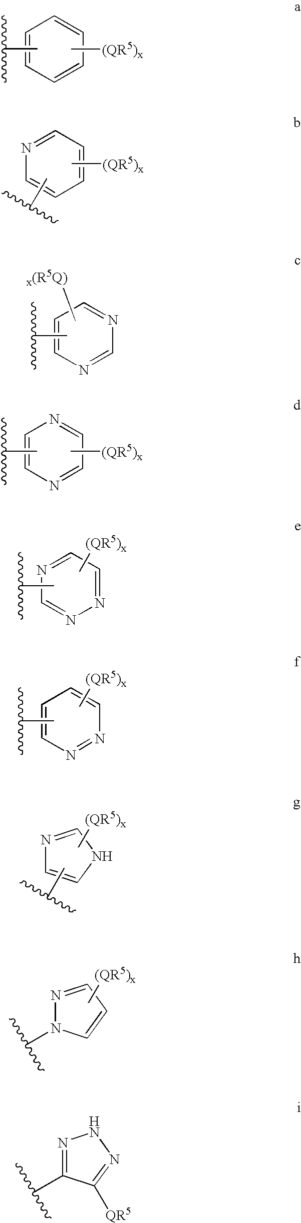 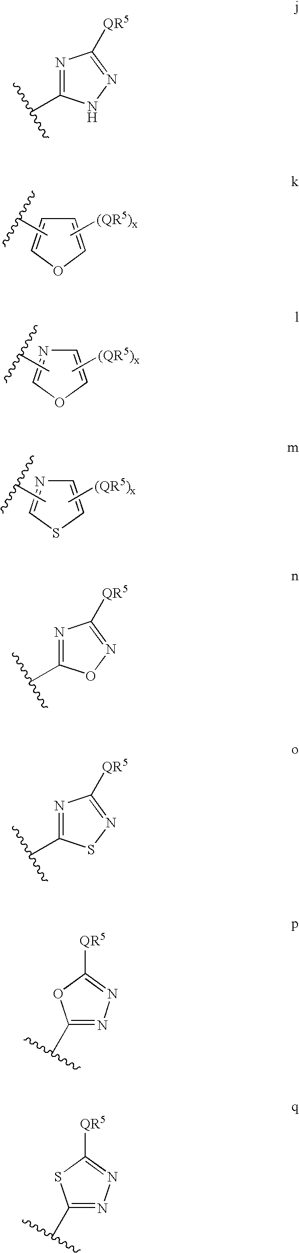 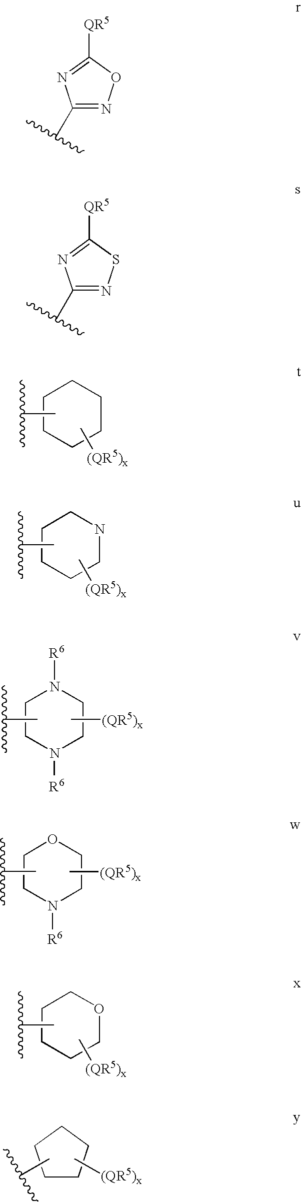 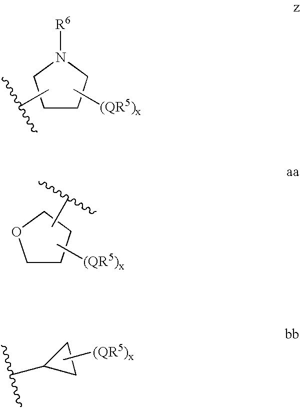
wherein Q and R⁵ are as defined generally above and in subsets herein, and x is 0-5.

21. The compound of claim 17, wherein Ar¹ is phenyl, pyrimidinyl, or pyridyl.

22. The compound of claim 17, wherein Ar¹ is phenyl and is substituted with two (x=2) or three (x=3) occurrences of Q—R⁵ and Ar¹ is: 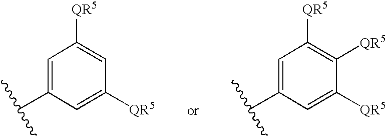
wherein each occurrence of QR⁵ is independently CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂.

23. The compound of claim 17, wherein Ar¹ is optionally substituted phenyl and compounds have one of formulas II-A-i, II-A-ii, II-B-i, II-B-ii, II-C-i, II-C-ii, II-D-i, or II-E-i: 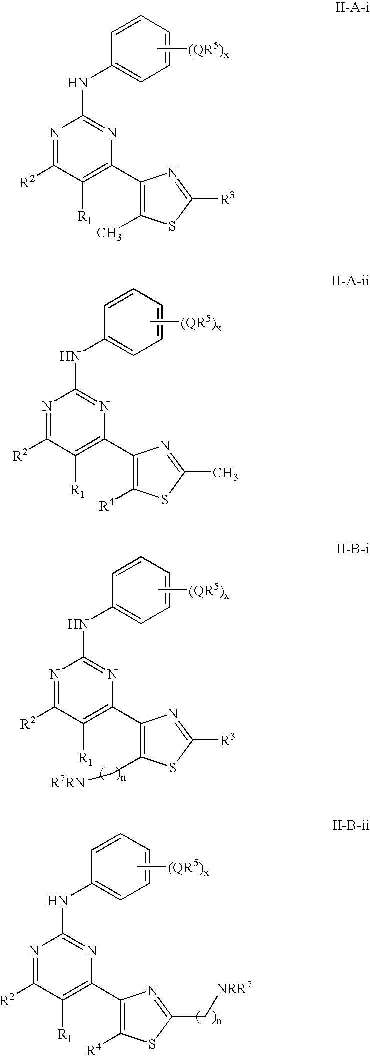 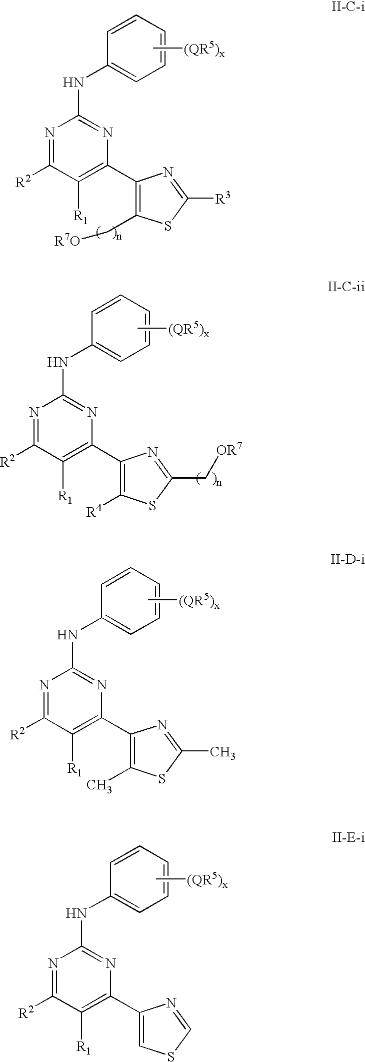 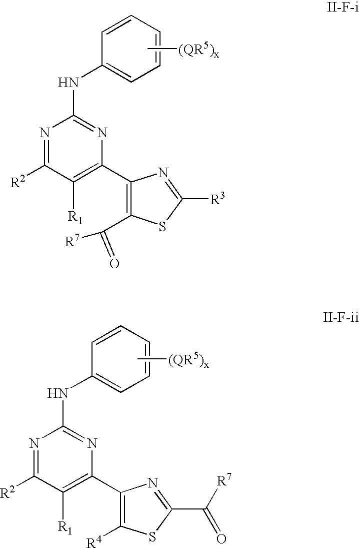
where x is 0-5.

24. The compound of claim 23, wherein each occurrence of Q is independently a bond or is an optionally substituted C₁-C₄ alkylidene chain wherein up to two non-adjacent methylene units of Q are optionally replaced by CO, CO₂, CONR, OCONR, NRCO, NRCO₂, NRSO₂, SO₂NR, O, S, or NR; and each occurrence of R⁵ is independently selected from R′, halogen, NO₂, CN, OR′, SR′, N(R′)₂, NR′C(O)R′, NR′C(O)N(R′)₂, NR′CO₂R′, C(O)R′, CO₂R′, OC(O)R′, C(O)N(R′)₂, OC(O)N(R′)₂, SOR′, SO₂R′, SO₂N(R′)₂, NR′SO₂R′, NR′SO₂N(R′)₂, PO(OR′)₂, C(O)C(O)R′, or C(O)CH₂C(O)R′, and x is 0, 1, 2, or 3.

25. The compound of claim 23, wherein each occurrence of Q—R⁵ is independently CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or two adjacent occurrences of Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur.

26. The compound of claim 23, wherein each occurrence of Q—R⁵ is independently fluoro, iodo, chloro, bromo, COCH₃, CO₂CH₃, C₁₋₄alkyl, NH₂, CH₂NH₂, NHMe, CH₂NHMe, N(Me)₂, CH₂N(Me)₂, N(Et)₂, CH₂N(Et)₂, NH(phenyl), CO(C₁₋₄alkyl), CH₂CO(C₁₋₄alkyl), NHCO(C₁₋₄alkyl), CH₂NHCO(C₁₋₄alkyl), CN, CH₂CN, OH, C₁₋₄alkoxy, optionally substituted benzyloxy, optionally substituted phenyloxy, CF₃, SO₂NH₂, SO₂NHMe, optionally substituted SO₂(phenyl), SO₂(C₁₋₄alkyl), CONH₂, CH₂PO(OR′)₂, or an optionally substituted group selected from a saturated, partially unsaturated, or fully unsaturated 5- or 6-membered ring having 0-3 heteroatoms independently selected from nitrogen, oxygen, or sulfur.

27. The compound of claim 23, wherein R¹ and R² are each independently hydrogen, N(R)₂, SR, OR, or TR, or R¹ and R², taken together form an optionally substituted saturated, partially unsaturated, or fully unsaturated 5-membered ring having 0-2 heteroatoms independently selected from N, O, or S.

28. The compound of claim 27, wherein R¹ and R² are each independently hydrogen, OH, CH₃, CH₂CH₃, OCH₃, CH₂OH, CH₂OCH₃, CH₂NH₂, CH₂NHCH₃, NH₂, or CH₂NH₂, or R¹ and R², taken together, form a fused optionally substituted pyrrolyl, pyrazolyl, or imidazolyl ring.

29. The compound of claim 23, wherein R³ is Z—R , wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is halogen, CN, N(R′)₂, NHCOR′, or R′.

30. The compound of claim 23, wherein R³ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1.

31. The compound of claim 23, wherein R⁴ is Z—R⁷, wherein Z is a bond or is an optionally substituted C₀₋₄ alkylidene chain wherein one methylene unit of Z is optionally replaced by O, NR, NRCO, NRCO₂, NRSO₂, CONR, C(O), C(O)O, and wherein R⁷ is selected from halogen, CN, N(R′)₂, NHCOR′, or R′.

32. The compound of claim 23, wherein R⁴ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1.

33. The compound of claim 23, wherein compounds have one of formulas II-A-i, I-B-i, II-C-i, or II-F-i and the compound variables are defined as:
a) x is 0, 1, or 2, and Q—R⁵ is CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur;
b) R¹ and R² are each independently hydrogen, N(R)₂, SR, OR, or TR, or R¹ and R², taken together form an optionally substituted saturated, partially unsaturated, or fully unsaturated 5-membered ring having 0-2 heteroatoms independently selected from N, O, or S; and
c) R³ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1.

34. The compound of claim 23, wherein compounds have one of formulas II-A-ii, II-B-ii, II-C-ii, or II-F-ii and one or more of the compound variables are defined as:
a) x is 0, 1, 2, or 3, and Q—R⁵ is CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur;
b) R¹ and R² are each independently hydrogen, N(R)₂, SR, OR, or TR, or R¹ and R², taken together form an optionally substituted saturated, partially unsaturated, or fully unsaturated 5-membered ring having 0-2 heteroatoms independently selected from N, O, or S; and
c) R⁴ is (CH₂)ₙhalogen, (CH₂)ₙCN, (CH₂)ₙOR⁷, (CH₂)ₙNRR⁷, (CH₂)ₙC(O)R⁷, (CH₂)ₙC(O)R⁷ (CH₂)ₙCH₃, (CH₂)ₙC(O)NRR⁷, (CH₂)ₙSR⁷, wherein R⁷ is (CH₂)ₘN(R′)₂, C₁-C₄alkyl, an optionally substituted 5- or 6-membered aryl, aralkyl, heteroaryl, or heteroaralkyl group, or R and R⁷, taken together with the nitrogen atom to which they are bound form an optionally substituted 3-8-membered saturated or partially unsaturated ring having 1-3 heteroatoms selected from nitrogen, oxygen, or sulfur, n is 0 or 1, and m is 0 or 1.

35. The compound of claim 23, wherein compounds have formula I-F-i, and one or more of the compound variables are defined as:
a) x is 0, 1, 2, or 3, and Q—R⁵ is CH₂halogen, halogen, CH₂CN, CN, CH₂CO₂R′, CO₂R′, CH₂COR′, COR′, R′, CH₂NO₂, NO₂, CH₂OR′, OR′, CH₂SR′, SR′, haloalkyl, CH₂SO₂N(R′)₂, SO₂N(R′)₂, CH₂N(R′)₂, N(R′)₂, NHCOR′, CH₂NHCOR′, CH₂PO(OR′)₂, PO(OR′)₂, or Q—R⁵, taken together with the atoms to which they are bound, form an optionally substituted saturated, partially unsaturated, or fully unsaturated 5-8-membered ring having 0-3 heteroatoms selected from nitrogen, oxygen, or sulfur; and
b) R¹ and R² are each independently hydrogen, N(R)₂, SR, OR, or TR, or R¹ and R², taken together form an optionally substituted saturated, partially unsaturated, or fully unsaturated 5-membered ring having 0-2 heteroatoms independently selected from N, O, or S.

36. The compound of claim 1, selected from:

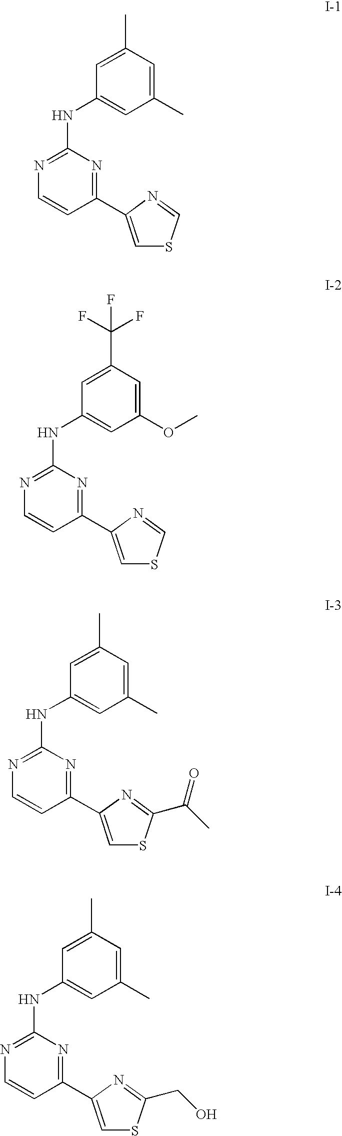

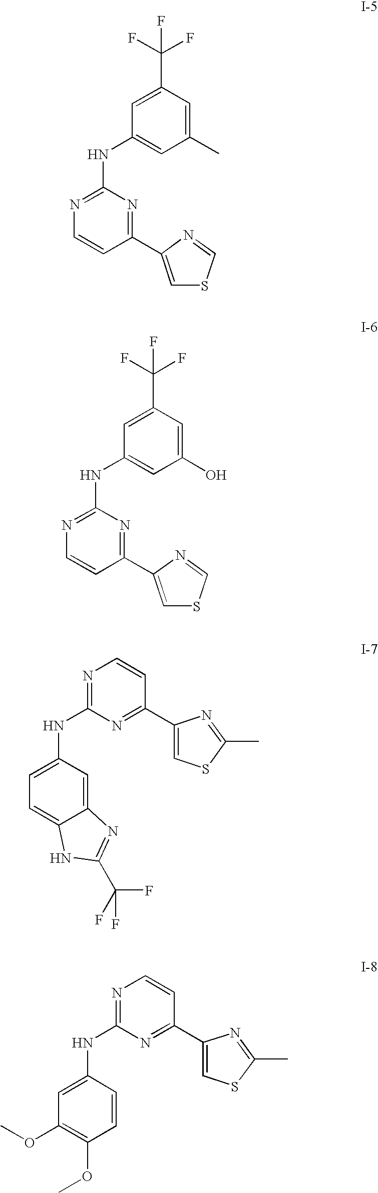

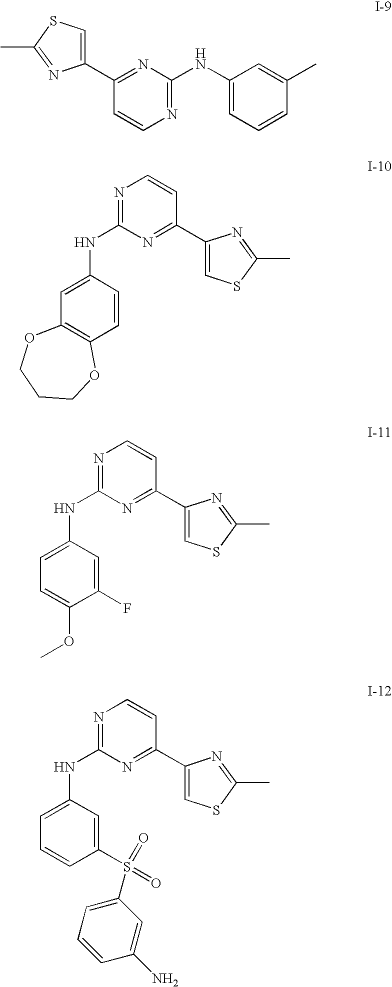

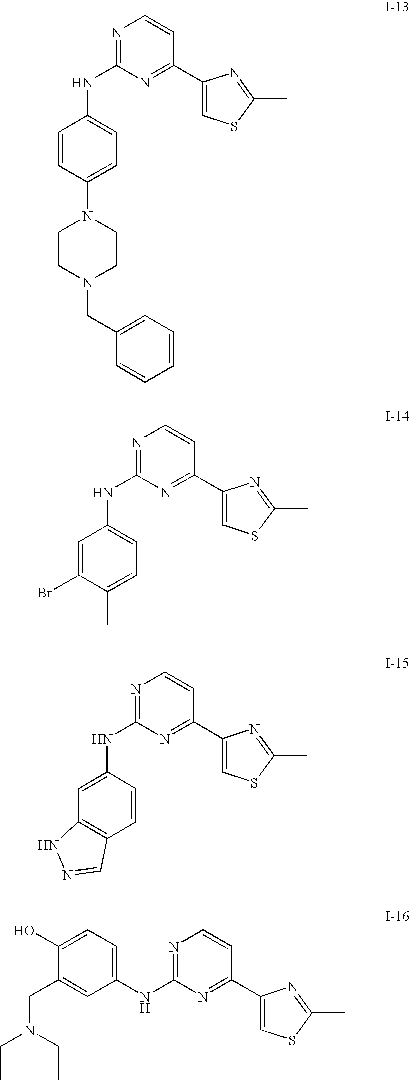

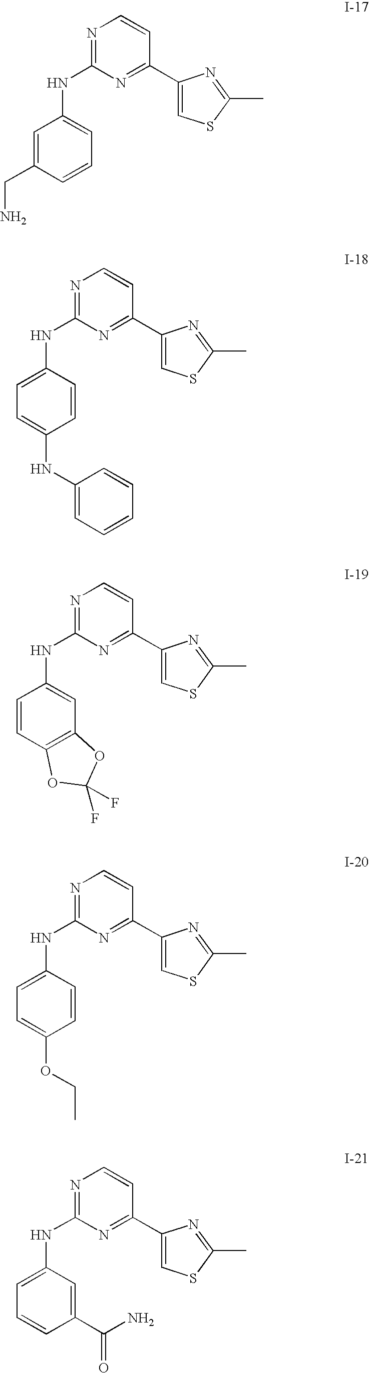

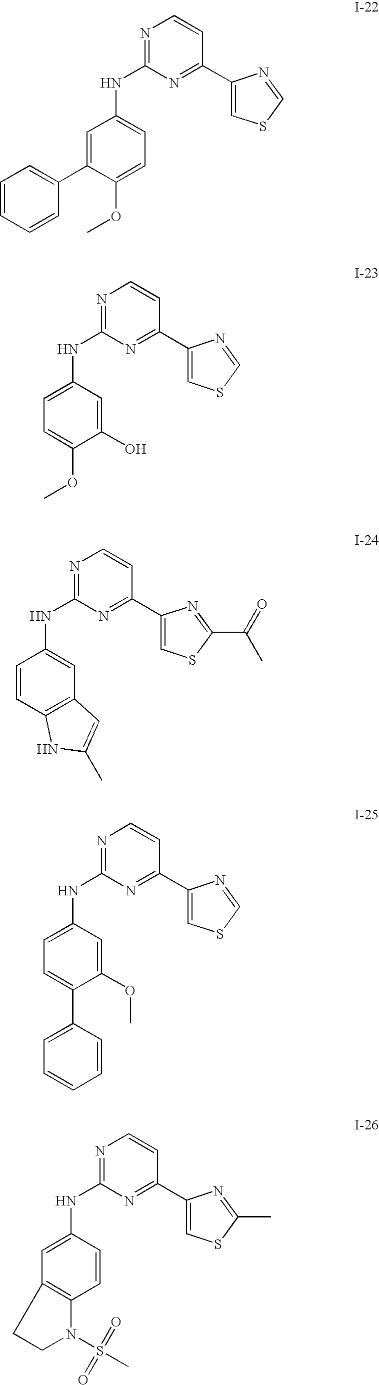

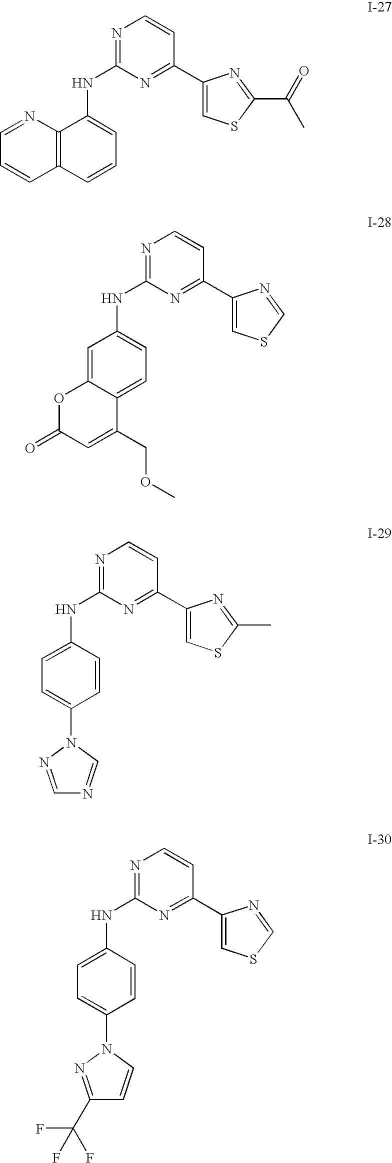

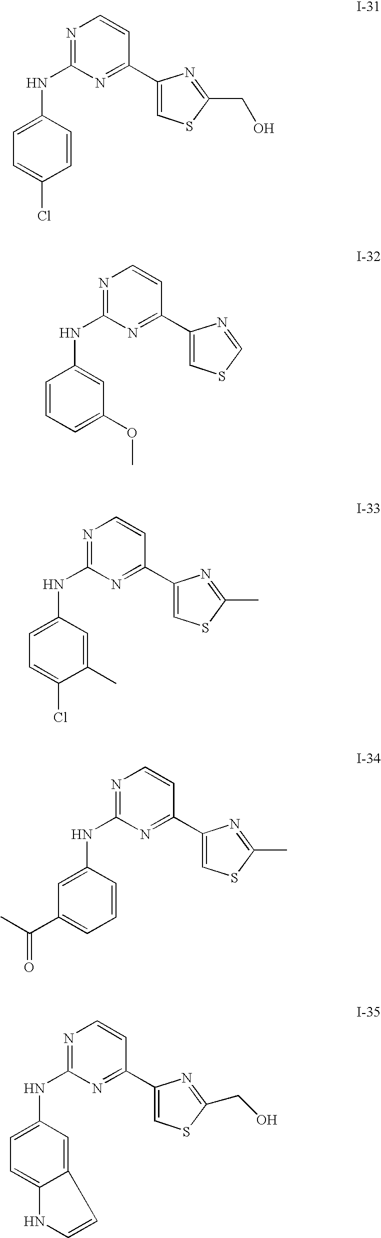

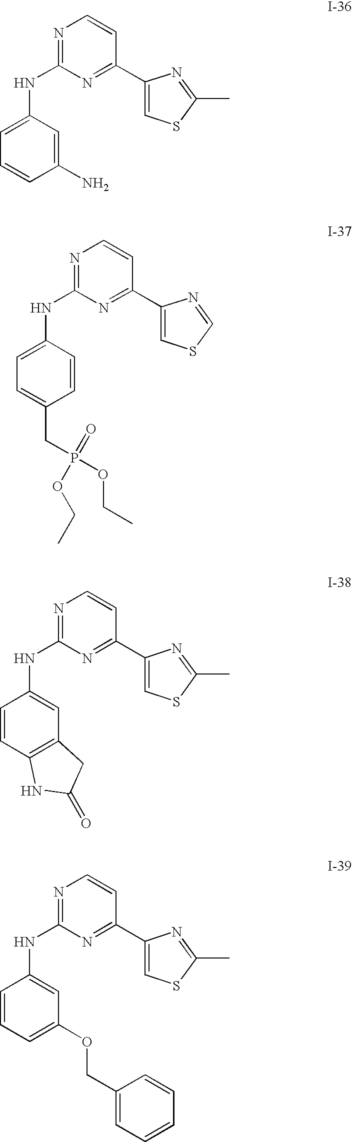

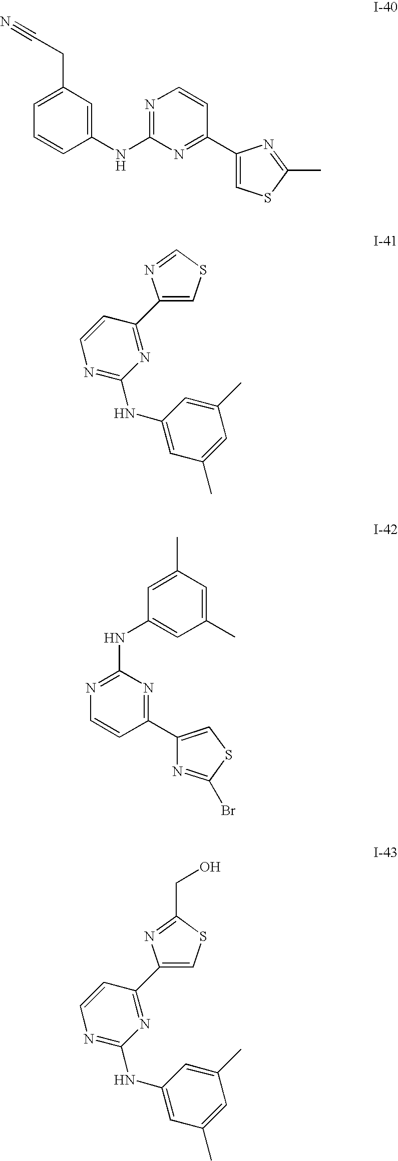

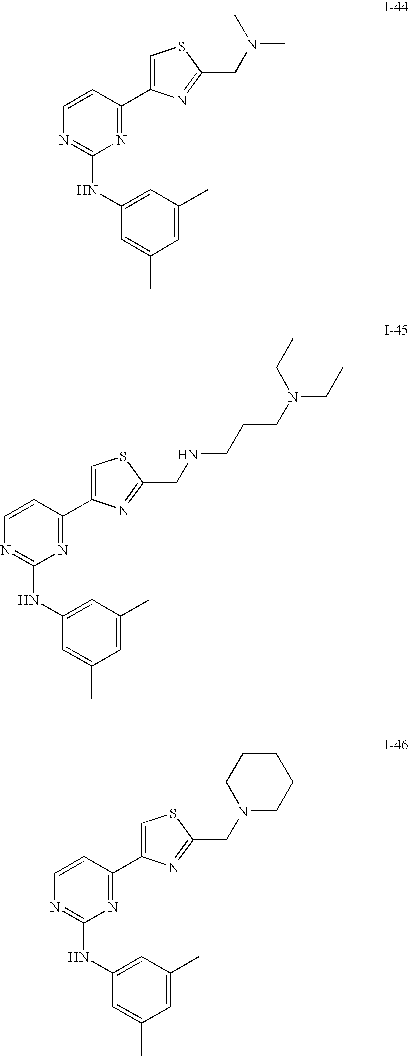

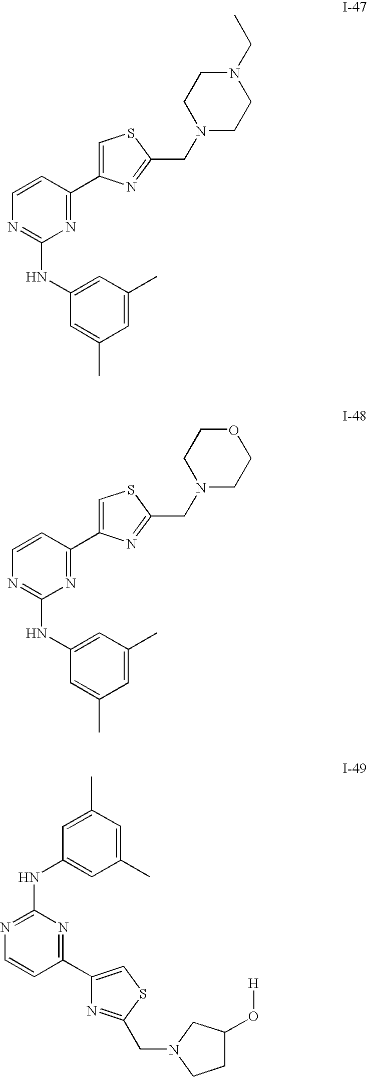

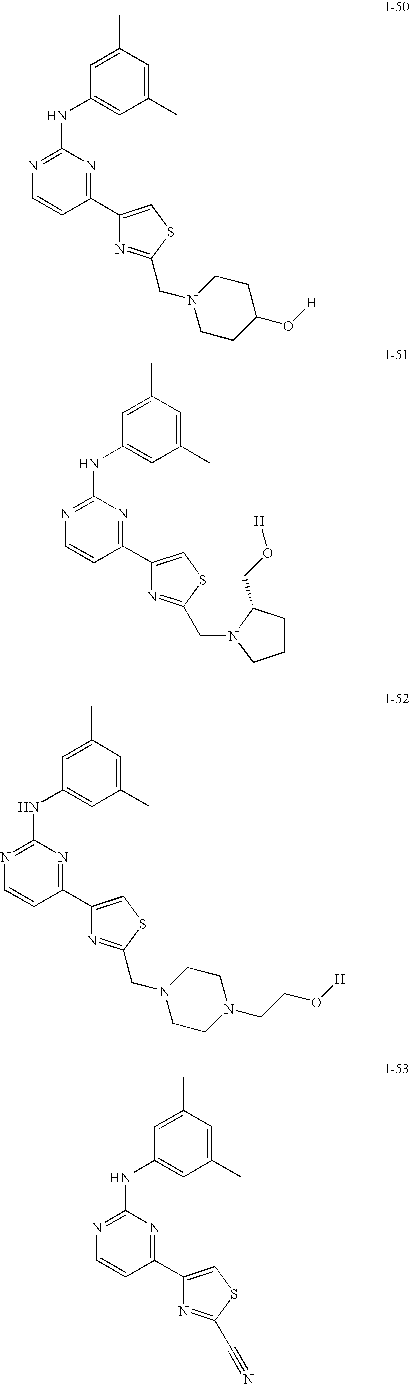

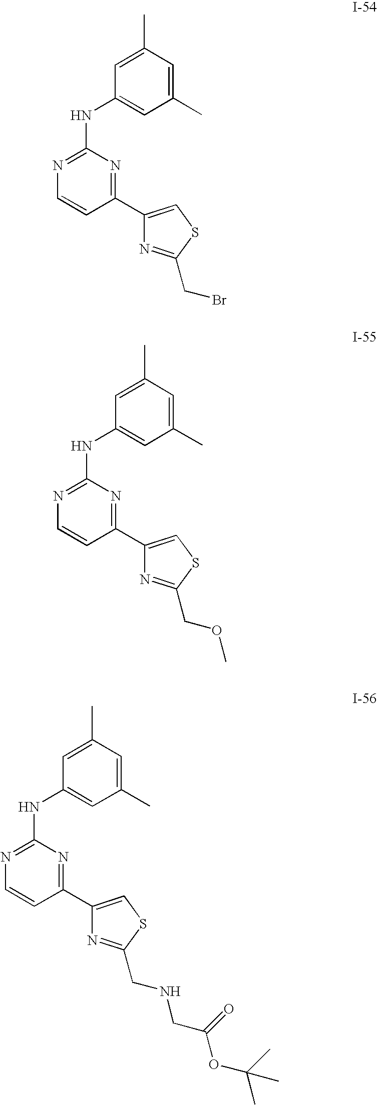

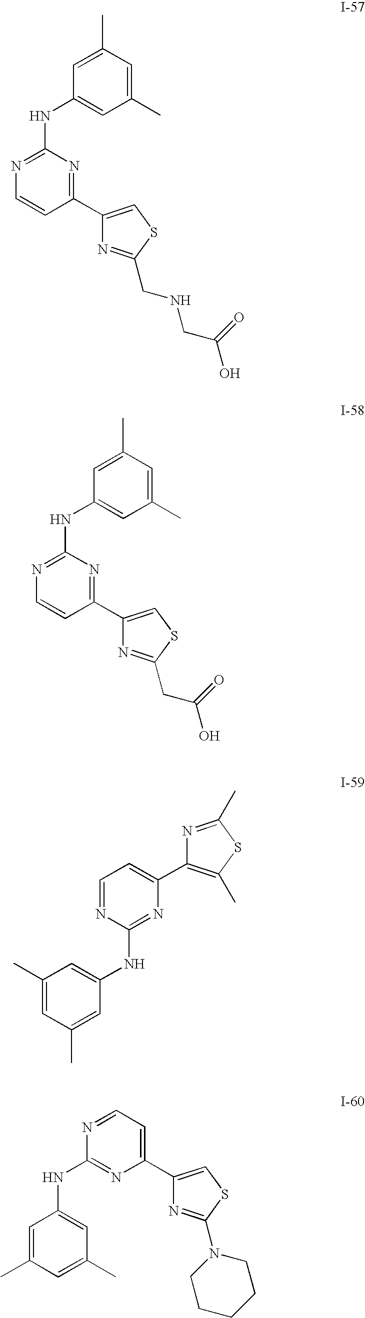

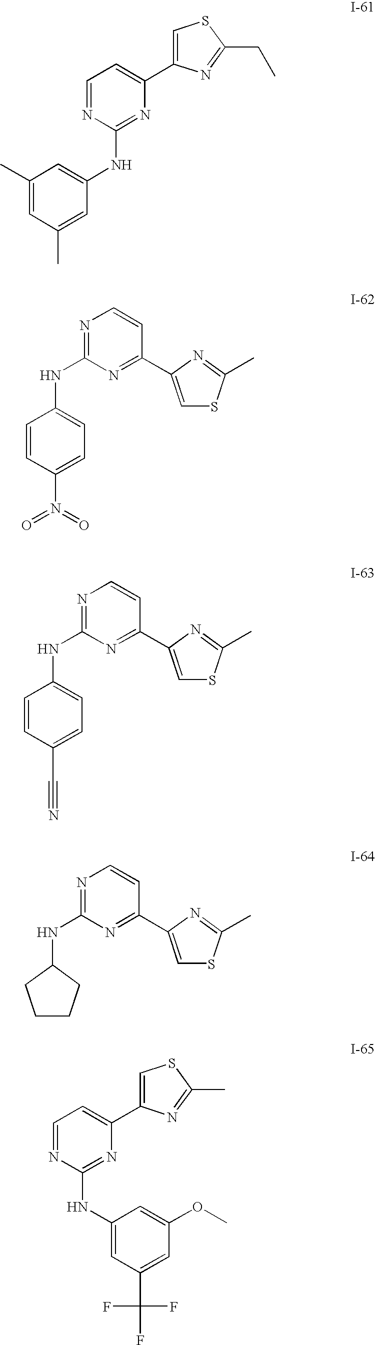

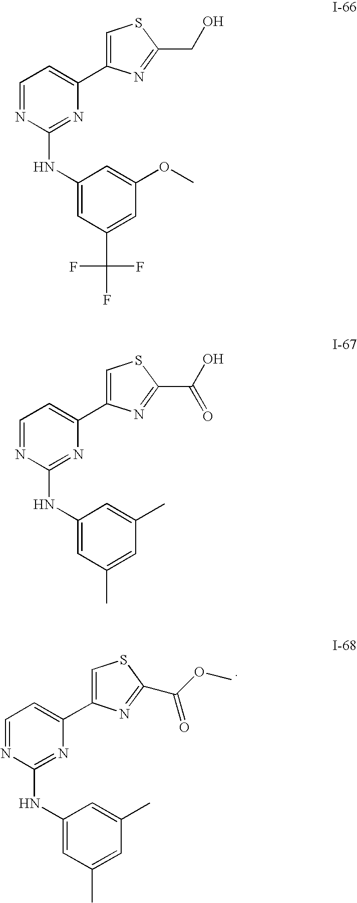

37. A composition comprising a compound of claim 1, and a pharmaceutically acceptable carrier, adjuvant, or vehicle.

38. The composition of claim 37, wherein the compound is in an amount to detectably inhibit SYK, or ZAP-70 protein kinase activity.

39. The composition of claim 37, additionally comprising a therapeutic agent selected from an anti-inflammatory agent, an anti-proliferative agent, an immunomodulatory or immunosuppressive agent, or an agent for treating immunodeficiency disorders.

40. A method of inhibiting SYK or ZAP-70 kinase activity in:
(a) a patient; or
(b) a biological sample;
which method comprises administering to said patient, or contacting said biological sample with:
a) a composition of claim 37; or
b) a compound of claim 1.

41. A method of treating or lessening the severity of treatment or lessening the severity of an immunodeficiency disorder, inflammatory disease, allergic disease, autoimmune disease, proliferative disorder, immunologically-mediated disease, or respiratory disorder, comprising the step of administering to said patient:
a) a composition of claim 37; or
b) a compound of claim 1.

42. The method according to claim 41, comprising the additional step of administering to said patient an additional therapeutic agent selected from an anti-inflammatory agent, an anti-proliferative agent, an immunomodulatory or immunosuppressive agent, or an agent for treating immunodeficiency disorders, wherein:
said additional therapeutic agent is appropriate for the disease being treated; and
said additional therapeutic agent is administered together with said composition as a single dosage form or separately from said composition as part of a multiple dosage form.

43. The method according to claim 41, wherein the disease is an immune disorder.

44. The method according to claim 41, wherein the disease is asthma.
